(19) United States
(12) Patent Application Publication
(10) Pub. No.: US 2015/0160222 A1
(43) Pub. Date: Jun. 11, 2015

(54) COMPOSITIONS AND METHODS FOR PROGNOSIS AND TREATMENT OF CANCER

(71) Applicant: NOVAZOI THERANOSTICS, Plano, TX (US)

(72) Inventors: Ritu Aneja, Lilburn, GA (US); Padmashree C.G. Rida, Plano, TX (US)

(21) Appl. No.: 14/559,593

(22) Filed: Dec. 3, 2014

Related U.S. Application Data
(60) Provisional application No. 61/912,467, filed on Dec. 5, 2013.

(51) Int. Cl. G01N 33/574; C12Q 1/68

(52) U.S. Cl. CPC G01N 33/57415; C12Q 1/6888; G01N 2333/47; G01N 2333/91205; G01N 2440/14; C12Q 2600/156

(57) ABSTRACT

A method of diagnosing and/or treating a patient diagnosed with breast cancer includes the steps of: (a) identifying a patient as having a triple negative breast cancer; (b) obtaining a sample from the triple negative breast cancer patient comprising breast cancer cells; and (c) determining whether the cells in the sample express an elevated level of nuclear HSET, wherein an elevated level indicates a poorer prognosis. The method may further include the step of determining whether the cells in the sample express elevated level(s) of one or more products upregulated with HSET, elevated levels of phosphorylated histone-H3 and/or exhibit enhanced Cdk1 activity. In certain embodiments, the method further includes the step of administering one or more therapeutic agents, such as HSET inhibitors, centrosome declustering agents, PARP inhibitors, Ras/MAPK pathway inhibitors, PI3K/AKT/mTOR pathway inhibitors or a combination thereof.

This application claims the benefit of U.S. Provisional Application Ser. No. 61/912,467, filed Dec. 5, 2013. The entirety of the aforementioned application is incorporated herein by reference.

This invention was made with government support from the National Cancer Institute at the National Institute of Health (NIH-NCI 1RO1CA169127-01). The government has certain rights in the invention.

### FIELD

The present invention generally relates to compositions, methods and kits for the prognosis and treatment of cancer and, in particular, triple negative breast cancer.

### BACKGROUND

Breast cancers are typically classified into several different subtypes: luminal A (ER positive and histologic low grade), luminal B (ER positive and histologic high grade), HER2 overexpressing, basal-like (2 types—BL1 and BL2), immunomodulatory (IM), mesenchymal (M), mesenchymal stem-like (MSL) and normal breast-like tumors.

Triple Negative Breast Cancer (TNBC) is a subtype of basal-like breast cancers characterized as estrogen receptor (ER) negative, progesterone receptor (PR) negative and human epidermal growth factor receptor 2 (HER2) negative based on immunohistochemistry (IHC) phenotype. TNBC is characterized by a unique molecular profile, aggressive nature, distinct metastatic patterns and lack of targeted therapies. It is estimated that approximately 170,000 cases of breast cancer worldwide are TNBC, which accounts for ˜10-20% of invasive breast cancers.

Clinical prognosticators for breast cancer include estrogen receptor (ER) status, progesterone receptor (PR) status, HER2 (human EGF receptor 2) status, the Nottingham Prognostic Index (NPI), the Ki67 Index, tumor grade, and clinical stage. In addition to ER, PR, and HER2 receptor status, clinicians use tumor grade and clinical stage to evaluate prognosis, albeit with very limited risk predictive accuracy.

Amplified centrosomes are widely recognized as a hallmark of cancer and, in particular, 80% of human breast tumors harbor supernumerary centrosomes. The presence of more than two centrosomes within a cell can pose a grave conundrum as it may lead to the assembly of a multipolar mitotic spindle and the production of nonviable progeny cells due to lethal levels of chromosomal loss (i.e., death-inducing, high-grade aneuploidy). However, cancer cells harboring extra centrosomes circumvent these catastrophic consequences and survive. This is achieved by centrosome clustering, whereby the excess centrosomes are artfully corralled into two polar foci to enable formation of a pseudo-bipolar mitotic spindle. During a preceding, transient, multipolar state, merotelic kinetochore-microtubule attachments occur, thus engendering low-grade, whole-chromosome missegregation that could be tumor-promoting.

HSET/KifC1, a minus end-directed motor protein that promotes microtubule cross-linking, sliding, bundling and spindle pole focusing, has been recently identified as an essential mediator of supernumerary centrosome clustering in cancer cells. HSET has also been shown to be indispensable for the clustering of acentrosomal microtubule organizing centers (MTOCs) whose production tends to be hyperactivated in cancer cells. By contrast, HSET function appears to be non-essential in healthy somatic cells due to the presence of two centrosomes that shoulder the responsibility of bipolar spindle assembly.

HSET's localization changes dynamically during cell cycle progression; HSET is sequestered in the nucleus in interphase, presumably to avoid untimely microtubule cross-linking. Upon nuclear envelope breakdown at the onset of mitosis, HSET is released into the cytoplasm to resume its activities in bipolar spindle biogenesis. During mitosis, HSET is localized both on the spindle poles and along the spindle length. With mitotic spindle breakdown in telophase, HSET is localized on the minus-end of microtubules near the spindle poles before being shuttled back into the nucleus. HSET transport inside the nucleus is regulated by Ran GTPase via association of the bipartite Nuclear Localization Signal of HSET with nuclear import receptors importin α/β.

Recent studies have focused on the association between HSET and malignancy. HSET is highly overexpressed in brain metastases, and its expression level in lung cancer is associated with increased risk of metastatic dissemination to the brain. Primary breast tumors also overexpress HSET as compared to matched normal breast tissue. Development of docetaxel resistance in breast cancer may be partly mediated by HSET. Its expression is upregulated in docetaxel-resistant breast tumors, and HSET-overexpressing MDA-MB-231 and MDA-MB-468 breast cancer cells (which are TN) exhibit enhanced survival compared to vector controls. In addition, MDA-MB-231 breast cancer cells rely on HSET for efficient clustering of supernumerary centrosomes, a process that not only suppresses potentially fatal spindle multipolarity but also facilitates low-grade chromosome missegregation during cell division. In fact, cells with supernumerary centrosomes rely on HSET-dependent centrosome clustering for their viability. HSET is required for centrosomal and acentrosomal spindle pole focusing in BT-549 breast cancer cells. Due to its intriguing association with malignancy, HSET presents a potential chemotherapeutic target for breast cancer patients, particularly those with triple negative breast cancer (TNBC).

Current treatment guidelines for breast cancer patients in the US (e.g., those of the NIH and NCCN) are based on clinical factors (e.g., age, menopausal status), tumor grade and stage, and the expression of prognostic and predictive markers (e.g., ER, PR, HER2). However, patients with similar clinicopathological features and a similar status with regard to conventional biomarkers may still respond differently to the same treatment, suggesting a need for better risk stratification schemes. To improve personalization of treatment regimens, more molecular biomarkers may be employed. While certain gene expression-based tests (specifically, OncotypeDx and Mammaprint) appear clinically valid for patients with ER+ breast cancer, their clinical utility remains controversial since modifying treatment based on their results may not improve outcomes. Furthermore, genomic tests continue to be expensive and technically challenging. Consequently, the need persists for protein biomarkers, which can be assessed via relatively inexpensive, facile immunohistochemical assays. A novel panel, Mammostrat, successfully stratifies patients who take tamoxifen (because their tumors are ER+) by assessing five proteins, yet there remains a need to stratify ER-negative tumors, including TN breast cancers, which are confoundingly still characterized not by what they are but rather by what they are not. Further, clinical trials using new targeted therapies for triple negative breast cancer have achieved only limited success, perhaps due to the high heterogeneity of TN lesions and the necessity for better molecular stratification of this tumor class.

In light of the foregoing limitations there is a need for new biomarkers for triple negative breast cancer, particularly those of prognostic value, as well as new treatments for patients with triple negative breast cancer.

### SUMMARY

The present application is based, in part, on work with the protein HSET/KifC1, and features methods of assessing the prognosis or better predicting the outcome for a patient diagnosed with breast cancer. One aspect of the present application relates to a method of assessing the prognosis of a patient diagnosed with triple negative breast cancer, the method comprises the steps of performing an assay on a biological sample comprising breast cancer cells from the patient to determine whether the breast cancer cells express an elevated level of nuclear HSET; and providing an assessment of the prognosis of the patient based on the result of the assay, wherein an elevated level of nuclear HSET in the breast cancer cells indicates a poorer prognosis.

The method of determining whether the cells express an elevated level of nuclear HSET may be carried out by immunohistochemical analysis of a breast cancer sample or an analysis of a nuclear extract from the sample. In one embodiment, the immunohistochemical analysis involves exposing the sample to a monoclonal or polyclonal anti-HSET antibody under conditions sufficient to allow the antibody to specifically bind to HSET.

In another embodiment, the method further includes the step of determining whether the cells in the sample express elevated level(s) of one or more products that are upregulated with HSET, such as Ki67, survivin, phospho-survivin, HIF-1-alpha, and/or aurora kinase B, p-Bcl2, Mad1 or combinations thereof. Alternatively, or in addition, the method may include the step of determining whether the cells in the sample exhibit elevated levels of phosphorylated histone-H3, enhanced Cdk1 activity or both.

In certain embodiments, the method includes the step of identifying the patient as a person of African descent. In some instances, this can be carried out by determining the patient's geographic origin(s) by ancestry analysis of the patient's genomic DNA.

In a further aspect, the method includes administering an inhibitor of HSET to a patient found to express an elevated level of nuclear HSET. In certain embodiments, the inhibitor of HSET is a small molecule drug. The inhibitor of HSET may target the motor domain of HSET and/or may specifically bind to the HSET/microtubule binary complex and inhibit HSET microtubule-stimulated or microtubule-independent ATPase activity. In some embodiments, the inhibitor of HSET is a centrosome declustering agent selected from the group consisting of AZ82, PJ-34, griseofulvin, noscapine, 9-bromonoscapine, reduced bromonoscapine, N-(3-bromobenzyl) noscapine, aminonoscapine and CW069. In other embodiments, the inhibitor of HSET is an siRNA or an expression vector carrying an shRNA.

In some embodiments, the patient may be administered an HSET inhibitor in combination with an inhibitor of a product that is upregulated with HSET, such as Ki67, survivin, phospho-survivin, HIF1α, aurora kinase B, Mad1 and/or p-Bcl2.

In other embodiments, the patient may be administered an HSET inhibitor in combination with a PARP inhibitor, an inhibitor of the Ras/MAPK pathway, an inhibitor of the PI3K/AKT/mTOR pathway, an inhibitor of FoxM1 or Plk1 or Prc1, or a combination thereof.

In a further aspect, a kit for determining elevated expression of HSET includes an HSET binding agent along with one or more secondary binding agents specifically binding to one or more gene product(s) upregulated with HSET, such as Ki67, survivin, phospho-survivin, HIF-1-alpha, aurora kinase B, Mad1, p-Bcl2, FoxM1, Plk1 and Prc1. The kit may further include one or more reagents for staining of nuclei, and/or one or more reagents for preparation of a nuclear fraction or extract.

### DETAILED DESCRIPTION OF THE DRAWINGS

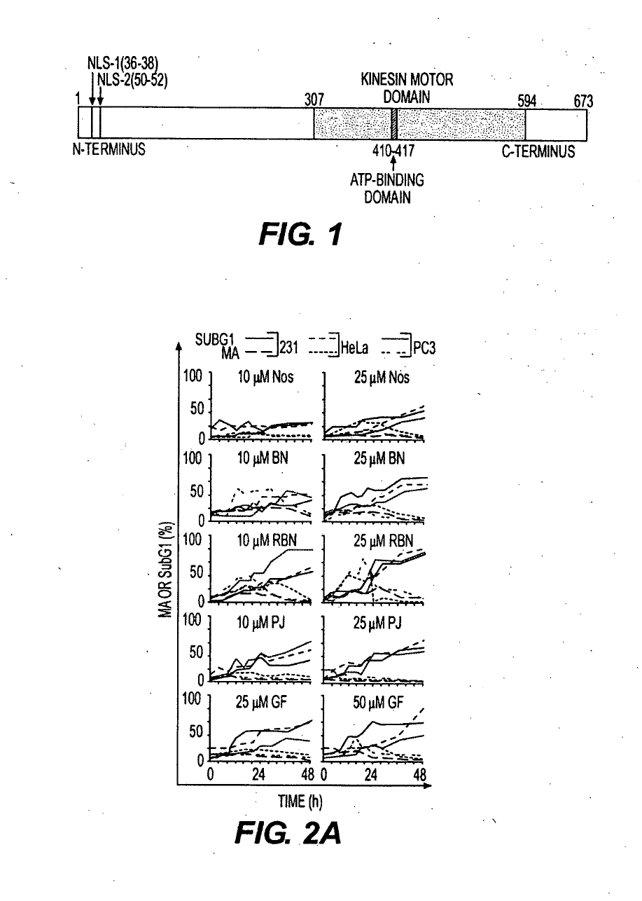

FIG. 1 is a schematic of HSET, identifying various regions and domains, including those targeted by anti-HSET antibodies.

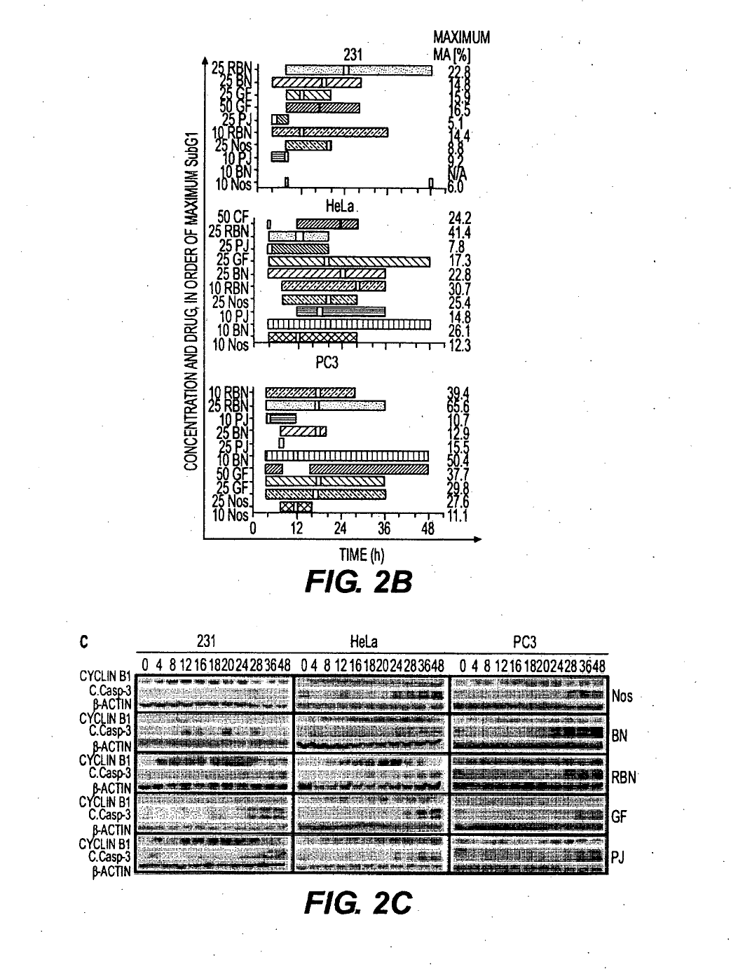

FIGS. 2A-2C depict mitotic arrest (MA) phenotypes observed upon treatment with putative centrosome declustering drugs. FIG. 2A shows subG1 and mitotically arrested cell population fractions with respect to time post-treatment with various putative declustering drugs. Declustering drugs included Nos, BN, RBN, PJ, and GF, all at 10 and 25 μM except GF, which was used at 25 and 50 μM, and cell lines included 231, PC3, and HeLa. These cell lines demonstrated differential susceptibility to various agents depending on drug concentration over the 48 h time period. In general, MA increased from 0 h to a peak near 24 h, followed by a decline in MA that coincided with increases in subG1 fractions. Results are representative of three independent experiments. FIG. 2B shows the duration of MA and peak MA by maximum subG1 fraction. Drugs are ranked in order of increasing peak subG1 from bottom to top along the y axis. The duration of MA (defined as the duration for which the mitotic population in drug-treated cells was greater than two times that in control cells) is plotted along the x axis. The time at which peak MA occurred is illustrated as a red bar and the value of peak MA is listed to the right of the graph. In 231 cells, 10 μM BN did not cause any MA; therefore, no bar is plotted. In 231 cells treated with 10 μM Nos in 231 cells and PC3 cells treated with 25 μM PJ, MA was observed at only one time point and is depicted using a single red bar. Some drugs produced a MA that then subsided and ultimately recurred, resulting in two bars being plotted, namely 50 μM GF in HeLa and PC3 cells. FIG. 2C shows western blot analysis of cell cycle-related proteins and caspase-3, a marker for apoptosis. To assess cell cycle progression following treatment with different declustering drugs (all at 25 μM), cell lysates were obtained at multiple time points over 48 h and immunoblotted for Cyclins E and B1. Increased levels of both cyclins compared with controls (0 h) were detected across cell lines with variable expression patterns depending on the drug and cell line. To evaluate apoptosis, cleaved caspase-3 (C. Caspase-3) was immunoblotted and eventual increases over controls were universally detected, typically by 24 h.

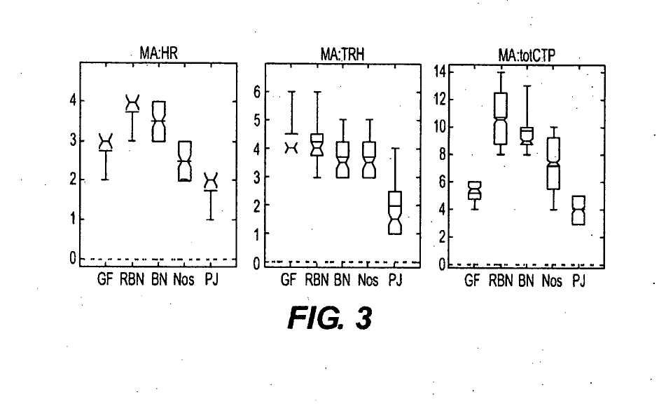

FIG. 3 depicts mitotic arrest metrics across cell lines for each declustering drug. For the box-and-whisker plots, the notch shows the median, box shows inter-quartile range, horizontal line shows mean, whiskers show min-max range. A lack of box in the plot occurs when the median is very close (or equal) to the inter-quartile range limits, in which case notch is shown with a default height and starting point of whisker line extension indicates 25% or 75% position. Because the coarse-grained data are integers and the size of the data sets are small (n<8), in some cases the median, lower or upper inter-quartile range values, or the max or min values, may coincide in some combination. This figure broadly visualizes clustering and correlation in the coarse-grained data. For instance, non-integer values have no intrinsic meaning but, for instance, a median value of 4.4 indicates a concentration of categories recorded in categories 4 or 5. Similarly, a positive R value above 0.5 suggests a possible positive correlation between the metrics versus a value near 0 or negative that would strongly suggest no correlation is likely. TRH=“time reach highest” value; CTP=“consecutive time points,” MA_totCTP=sum(MA_SnCTP for n=2 . . . 5).

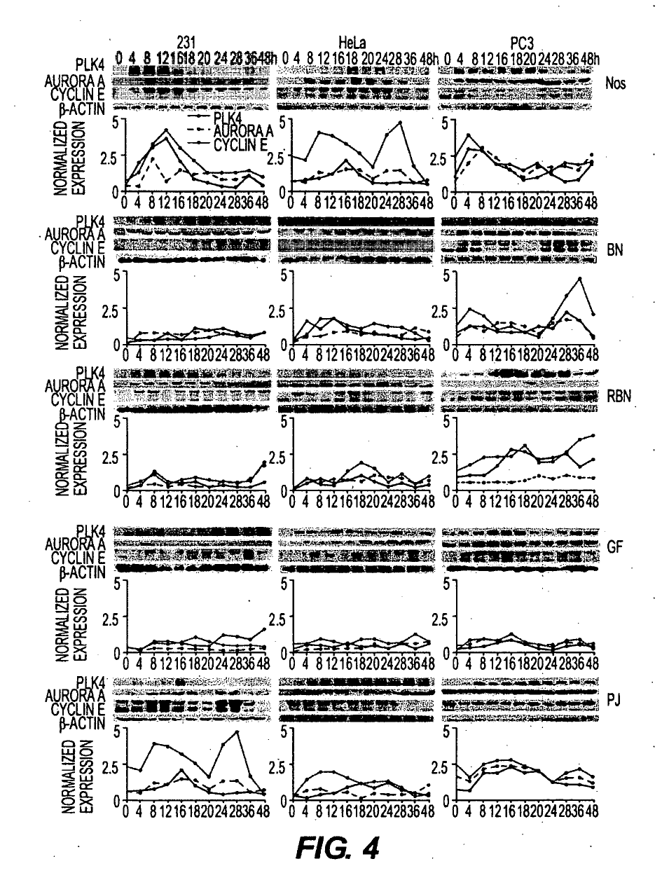

FIG. 4 shows centrosome declustering drug-induced changes in expression levels of markers of centrosome amplification. To evaluate the levels of centrosome amplification (CA) markers upon treatment with declustering drugs at a concentration of 25 μM, the levels of PLK4, Cyclin E, and Aurora A were assessed by western blotting, revealing eventual increases over untreated controls across cell lines. Increases in expression levels of PLK4 and Aurora A were generally rapid, often appearing by 4 h. Levels tended to vary thereafter depending on the drug and cell line. Densitometry was performed to quantitate the changes in levels of CA markers relative to β-actin over time, and the changes in actin-normalized expression levels over the time-course of the experiment are depicted graphically beneath each sets of blots. As the Cyclin E blots revealed two closely placed bands (49 and 43 kDa) corresponding to the two spliced forms, the Cyclin E band intensity was generated as a sum of the two band intensities.

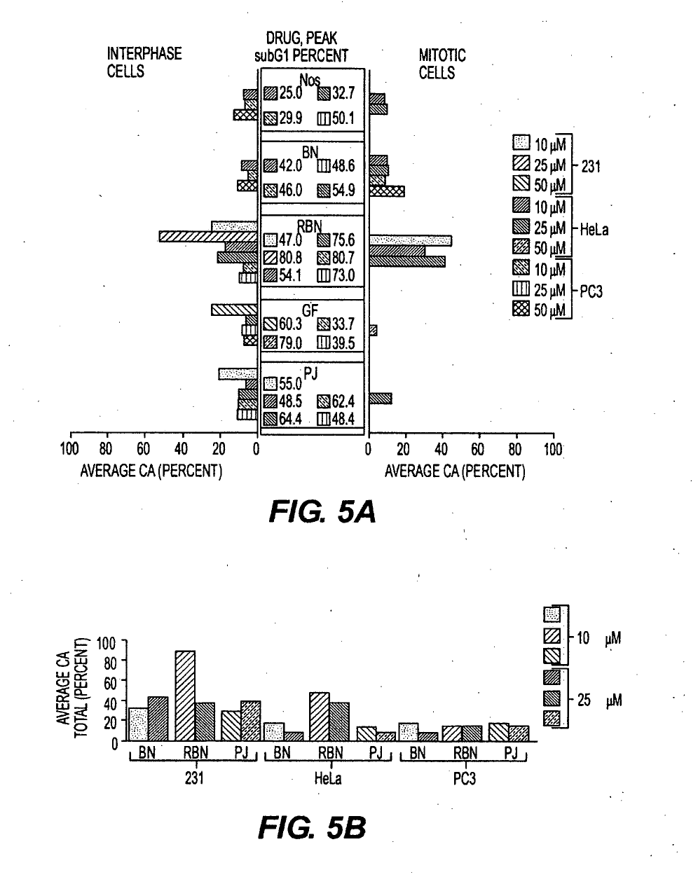

FIGS. 5A-5B show average CA observed over 24 h and its relationship with peak subG1 for each drug treatment regimen. FIG. 5A displays only statistically significant (P<0.05) increases in average CA over controls. To calculate average CA, the sum of percentage of (interphase or mitotic) cells showing CA at the 6, 12, 18, and 24 h time points was divided by 4. FIG. 5B depicts the sum of average CA (interphase plus mitotic) observed when 231 cells were treated with RBN, BN, and PJ, compared with the treatment of HeLa and PC3 cells with the same three drugs.

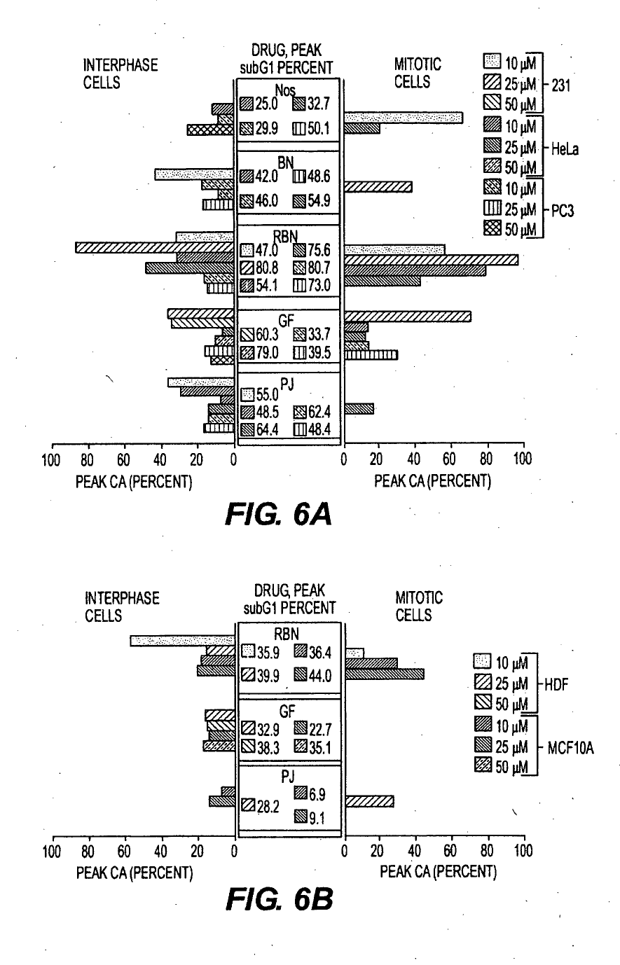

FIGS. 6A-6B show peak induction of CA and subG1 in cancer cell (FIG. 6A) and non-malignant (FIG. 6B) cell lines. Only statistically significant changed values are depicted.

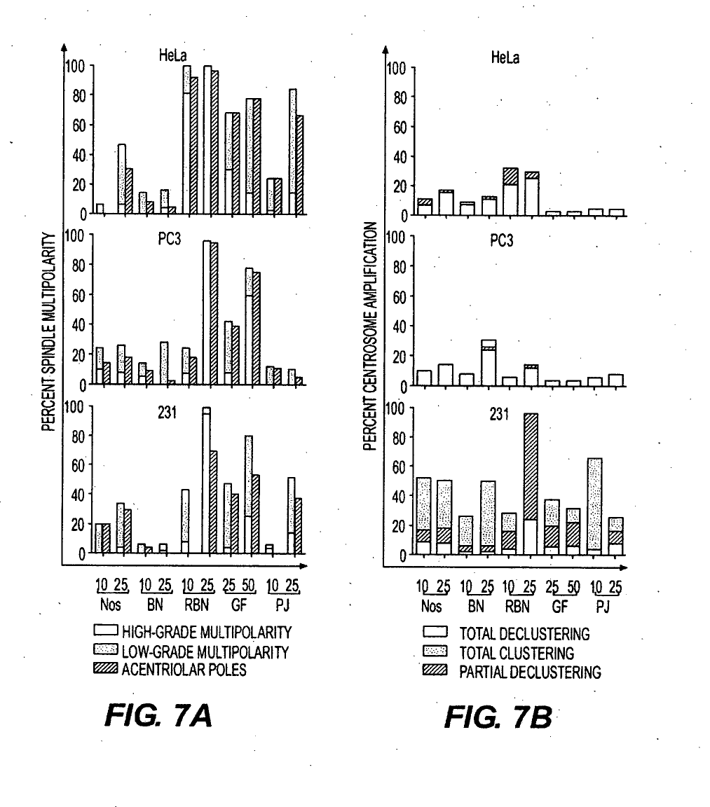

FIG. 7A shows peak spindle multipolarity (MP) and peak acentriolar pole formation induced by different declustering drugs in 231, HeLa, and PC3 cells. The maximum extents of MP induction of high grades (5+ poles) and low grades (3-4 poles) and acentriolar pole formation (at least one pole without centrioles) across a 24-h period are given for all drugs. FIG. 7B shows peak CA and declustering of amplified centrosomes induced in 231, HeLa, and PC3 cells. The maximum extent of CA in mitosis over 24 h is depicted by the height of the bar. The extent of total clustering (all centrosomes clustered at two poles), total declustering (all centrosomes separated to different poles), and partial declustering (one or more poles with 2+ centrosomes) are given for that same time point.

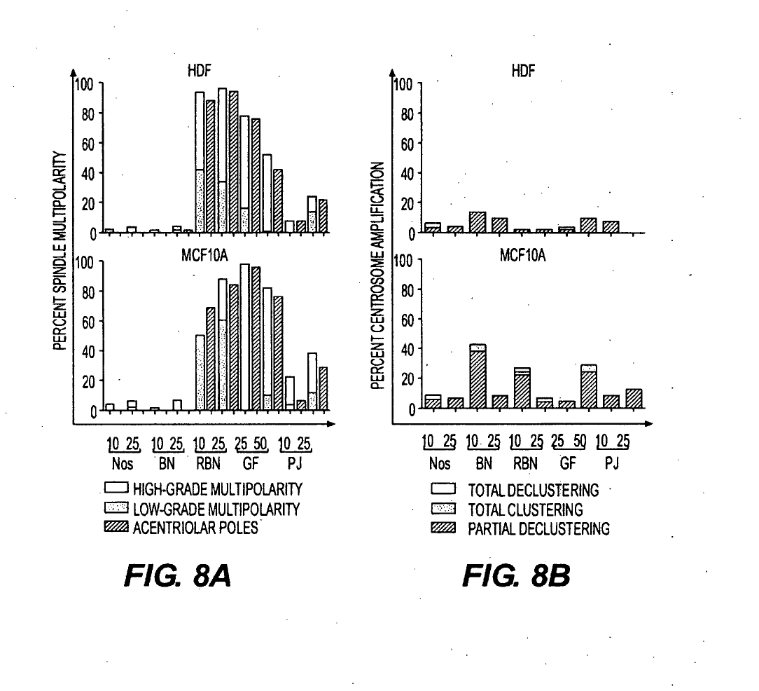

FIG. 8A shows induction of peak MP and peak acentriolar pole formation by different declustering drugs in human dermal fibroblasts (HDFs) and MCF10A cells. The maximum extents of MP induction of high grades (5+ poles) and low grades (3-4 poles) and acentriolar pole formation (at least one pole without centrioles) across a 24 h period are given for all drugs. FIG. 8B shows induction of peak CA and declustering of amplified centrosomes in HDFs and MCF10A cells. The maximum extent of CA in mitosis over 24 h is depicted by the height of the bar. The extent of total clustering (all centrosomes clustered at two poles), total declustering (all centrosomes separated to different poles), and partial declustering (one or more poles with 2+ centrosomes) are given for that same time point.

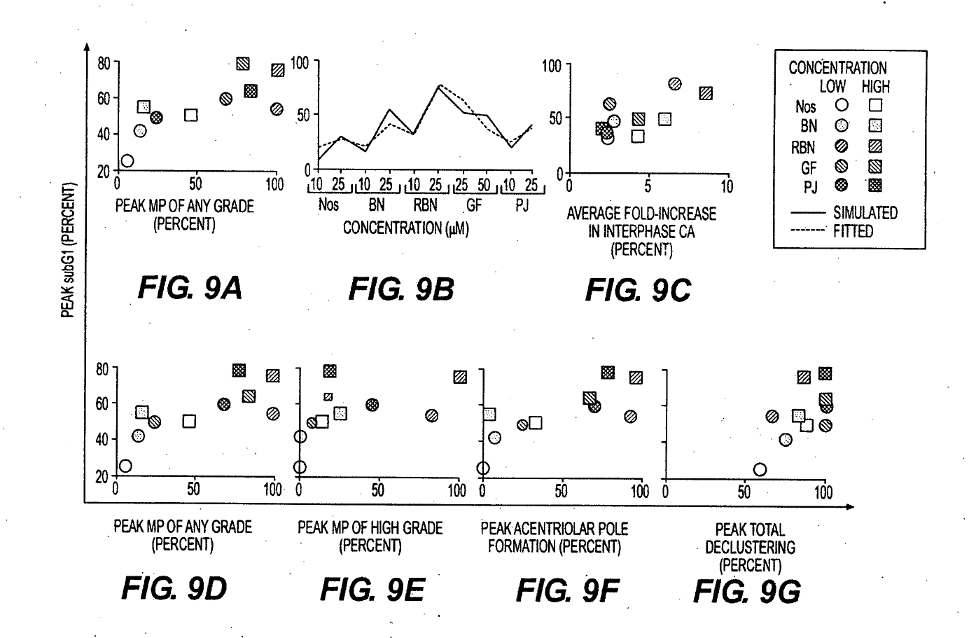

FIGS. 9A and 9B show correlates of peak subG1 percent in 231 cells by beta regression. FIG. 9A demonstrates a clear trend for increasing peak MP of any grade and peak subG1, which was highly statistically significant (P=0.006; pseudoR²=0.833). FIG. 9B shows that multiple regression using peak MP (high grade) and peak MP (low grade) produced an even better, statistically significant fit (red line) compared with simulated values (P=0.001; pseudoR²=0.860). Within this model, both variables were very highly statistically significant (P<0.0001), with peak high-grade MP showing a positive correlation and peak low-grade MP showing a negative correlation with peak subG1 (based on the sign of the beta coefficients). FIG. 9C shows correlates of peak subG1 percent in PC3 cells by linear regression. In these cells, the average fold increase in interphase CA shows some association with peak subG1, which almost reached statistical significance and which produced a good fit (P=0.057; R²=0.619). FIGS. 9D to 9G show correlates of peak subG1 percent in HeLa cells by beta regression. FIG. 9D shows that increasing peak MP of any grade was associated with peak subG1 (P=0.0055; pseudoR²=0.575), as was the case in FIG. 9E, which shows increasing peak MP of high grade (P=0.028; pseudoR²=0.271). FIG. 9F shows increasing peak acentriolar pole formation (P=0.0023; pseudoR²=0.600), and FIG. 9G shows increasing peak total declustering (P=0.020; pseudoR²=0.424).

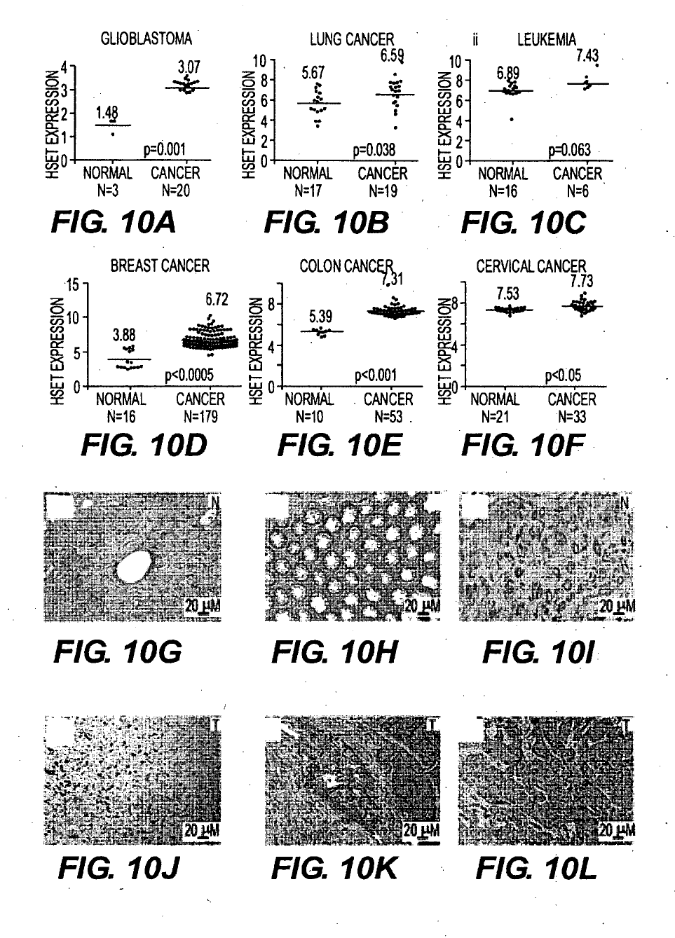

FIGS. 10A-10F show scatter plots depicting HSET gene expression in normal (green dots) versus tumor (red dots) tissues in (FIG. 10A) glioblastoma, (FIG. 10B) lung carcinoma, (FIG. 10C) leukemia, (FIG. 10D) breast carcinoma, (FIG. 10E) colon carcinoma and (FIG. 10F) cervical carcinoma. Data were obtained from one-channel microarrays available from the GEO database. Robust multiarray normalization was performed to obtain the differences depicted in the plots. FIGS. 10G-10L are immunohistographs showing HSET expression in glioblastoma tissue where a representative normal tissue (N) (FIG. 10G) is compared to tumor tissue (T) (FIG. 10J); in colon tumor (FIG. 10K) versus adjacent normal (FIG. 10H) tissue; and in cervical tumor (FIG. 10L) versus adjacent normal (FIG. 10I) tissue.

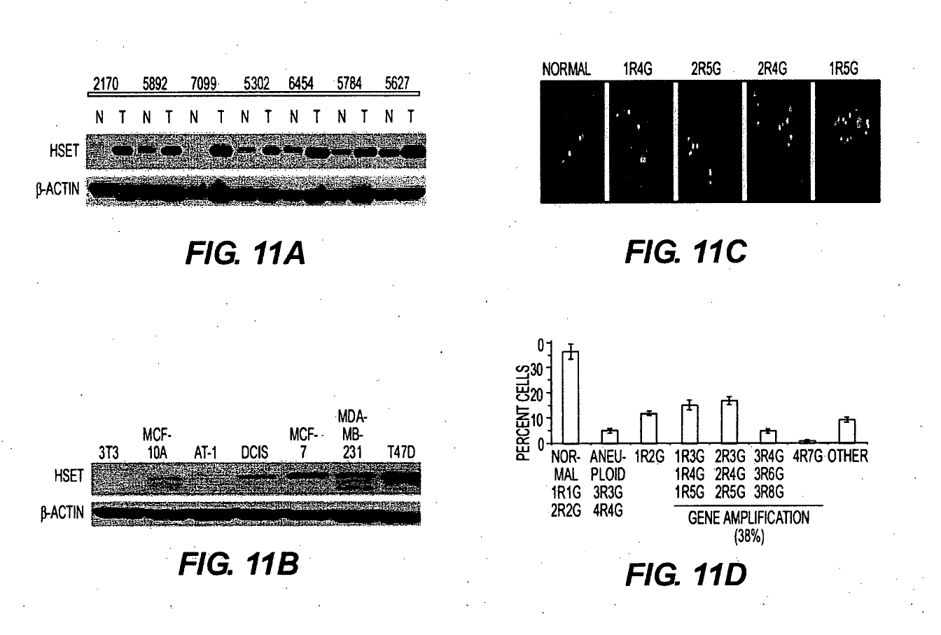

FIGS. 11A-11D show HEST gene expression in breast cancer tissues. FIG. 11A shows an analysis of HSET protein expression by western blotting of (A) cell lystates from 16 paired clinical breast tumor tissues (T) and normal adjacent tissues (N). Representative results of 7 paired samples are shown. FIG. 11B shows an immunoblot analysis of HSET expression in an MCF10A series of cell lines representing a continuum from near-normal breast (MCF-10A) to pre-malignant (MCF10-AT1) to comedo ductal carcinoma in situ (MCF10-DCIS), as well as aggressive breast cancer cell lines, such as MDA-MD-231 and T47D and the normal mouse fibroblast cell line, 3T3. FIG. 11C shows representative confocal micrographs depicting fluorescence in situ hybridization of two bacterial artificial chromosome probes to paraffin-embedded primary breast tumor tissues, one from the HSET locus on chromosome 6 (RPCI-11 602P21, green) and one from the chromosome 6 centromere (CH514-7B4, red). FIG. 11D shows amplifications of HSET visualized as an increase in the number of green signals (denoted as G) relative to the number of red control centromere signals (denoted as R), where 1R1G and 2R2G represent normal HSET gene copy numbers, and 1R4G, 2R4G, 2R5G, 1R5G, etc. represent instances where the HSET gene locus is amplified. FIG. 11D is a bar graph representation of various combinations of red and green signals observed for the HSET locus and chromosome 6 centromere as determined by visual quantitation from confocal images. 1R1G and 2R2G are considered normal copy numbers, elevated copy numbers with the same ratio of R and G signals are considered aneuploid (3R3G, 4R4G) and all other combinations with higher G-to-R ratios are considered as representing instances where the HSET gene is amplified.

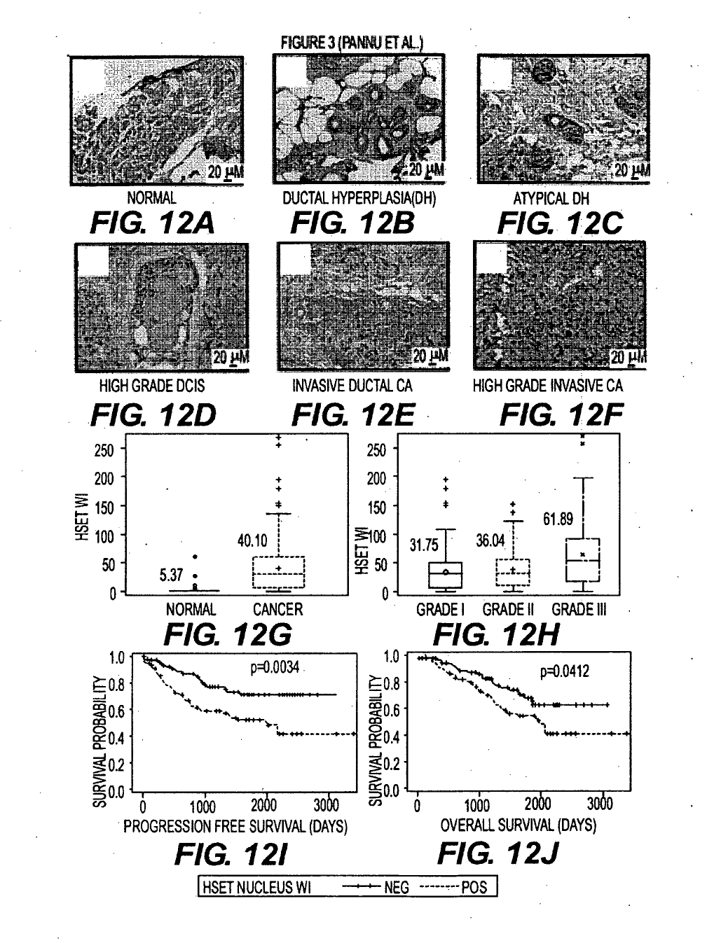

FIGS. 12A-12F depict immunohistographs showing HSET expression in (FIG. 12A) normal breast, (FIG. 12B) ductal hyperplasia, (FIG. 12C) atypical ductal hyperplasia, (FIG. 12D) ductal carcinoma in-situ, (FIG. 12E) invasive ductal carcinoma, low-grade and (FIG. 12F) invasive ductal carcinoma, high-grade. Brown (DAB) color shows HSET staining. Intensities of nuclear HSET staining were quantified using image analysis Aperio Image Scope v.6.25 software. A weighted index (WI) for HSET expression was calculated and was assessed in 384 breast cancer and 19 normal samples. FIGS. 12G and 12H depict box-and-whisker plots showing the (FIG. 12G) HSET WI in normal breast and tumor samples and (FIG. 12H) HSET WI across Grade I (n=40), Grade II (n=237) and Grade III (n=62) breast cancer samples. FIG. 12I shows the probability of progression-free survival of 163 breast cancer patients with HSET nuclear expression above or below the median HSET WI value, referred to as positive and negative, respectively (p=0.0034); FIG. 12J shows the probability of overall survival of 163 patients with positive and negative HSET WI (p=0.0412). Statistical analysis was conducted using SAS Version 9.3. Scale bar=10 μm. Red arrows indicate positive nuclear HSET staining.

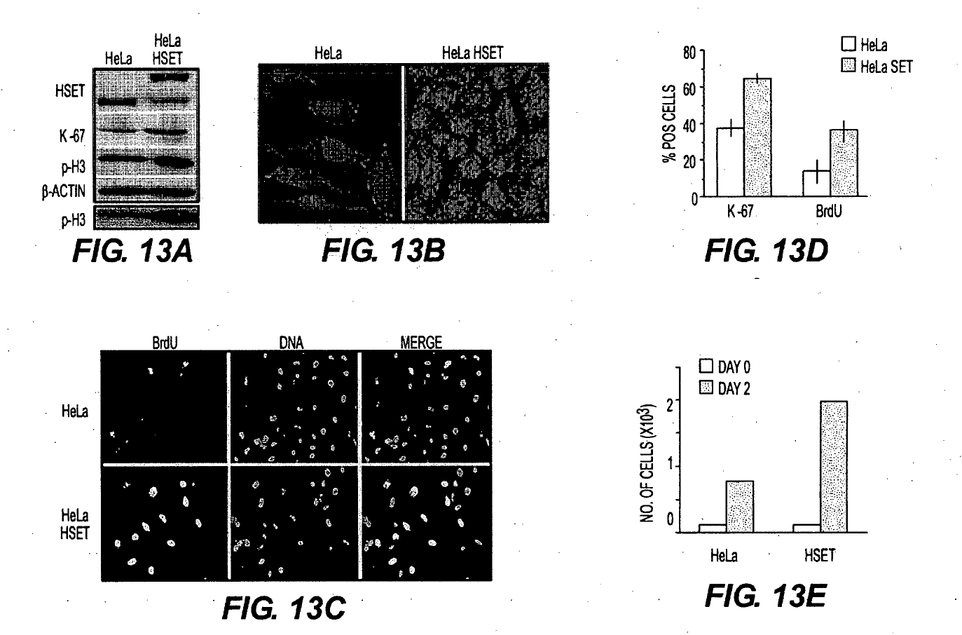

FIGS. 13A-13E show cell proliferation in HeLa cells. FIG. 13A depicts immunoblots showing higher Ki67 and p-Histone H3 in HeLa-HSET-GFP (denoted as HeLa HSET) cells as compared with HeLa cells. A kinase activity assay showed higher cdk1 activity in HeLa-HSET-GFP cells as reflected in enhanced phosphorylation of Histone H3 by cdk1 as compared to HeLa cells. The two bands representing HSET expression correspond to the endogenous HSET levels (lower band) and the GFP-HSET levels (upper band). FIG. 13B depicts confocal immunomicrographs showing higher Ki-67 expression (red) in HeLa-HSET-GFP cells as compared with HeLa cells. FIG. 13C depicts immunofluorescence images showing higher BrdU incorporation in HeLa-HSET-GFP cells as compared to HeLa cells. Randomly dividing HeLa-HSET-GFP and HeLa cells were incorporated with BrdU and immunostained with anti-BrdU antibody (green) to visualize the cells traversing S phase. FIG. 13D shows bar graphs depicting the percentage of cells that are Ki-67 or BrdU positive in HeLa and HeLa-HSET cells. FIG. 13E shows bar graphs representing the number of cells in the cell proliferation assay counted by Trypan Blue at Day 0 and Day 2 of seeding.

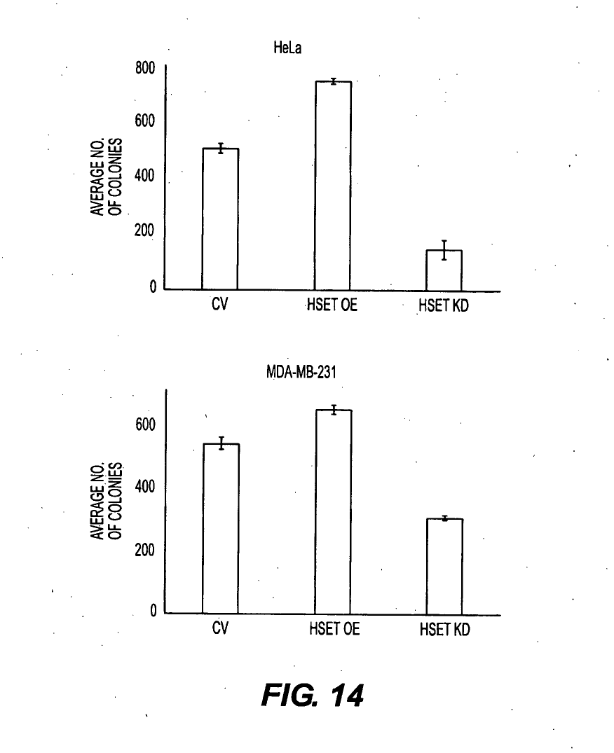

FIG. 14 shows bar graphs pertaining to colony formation assays in HeLa and MDA-MB-231 cells with HSET OE and KD. The bar graphs represent average number of colonies counted 72 h after the transfected cells were seeded (2000 cells per well). Cells were stained with crystal violet, colonies were counted manually and the average of 3 wells was plotted in the bar graphs (p<0.005).

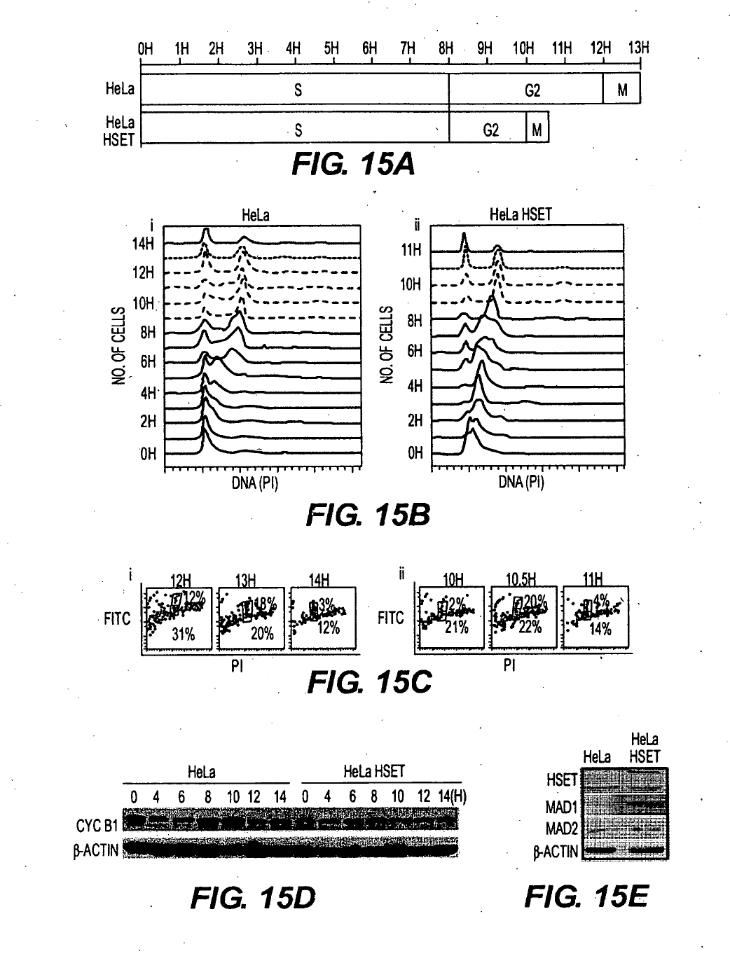

FIGS. 15A-15D show that HEST overexpression accelerates cell cycle kinetics. FIG. 15A shows cell cycle histograms representing cell cycle profiles of synchronized HeLa and HeLa-HSET-GFP cells from the point of thymidine block release (0 h) to the point after mitotic exit (14 h and 11 h, respectively). FIG. 15B shows FACS profiles of (i) HeLa and (ii) HeLa-Hset cells showing their DNA content distribution at various time-points (indicated on y-axis) after release from single thymidine block (synchronization at the G1/S boundary). FIG. 15C shows dot plots of PI (DNA) vs FITC (MPM-2) showing cells in G2 (lower box) and M phase (upper box) specifically during the time of mitotic exit in (i) HeLa and (ii) HeLa-HSET-GFP cells. The two-color scatter plot (PI vs. GFP) shows two box gates, where the lower box represents the G2 population (PI-4N and FITC negative) and the upper box represents the M population (PI-4N and FITC positive). The G2/M population is represented by double the intensity of PI (4N) as compared with the G1 population (2N). Mouse anti-MPM-2 antibody tagged with anti-mouse Alexa-488 secondary antibody was used as a mitosis-specific marker, to distinguish G2 and M populations. The time for mitotic exit was determined by assessing the population in the upper gate of the 2-color scatter plot. A sudden surge in the proportion of mitotic cells followed by a rapid fall indicates the time of mitotic exit. The time of mitotic exit for HeLa cells was determined to be 13 h, whereas 10.5 h was the time of mitotic exit for HeLa-HSET-GFP cells. FIG. 15D depicts immunoblots showing cyclin B1 protein levels in synchronized HeLa and HeLa-HSET-GFP cells following release from thymidine block at the G1/S boundary.

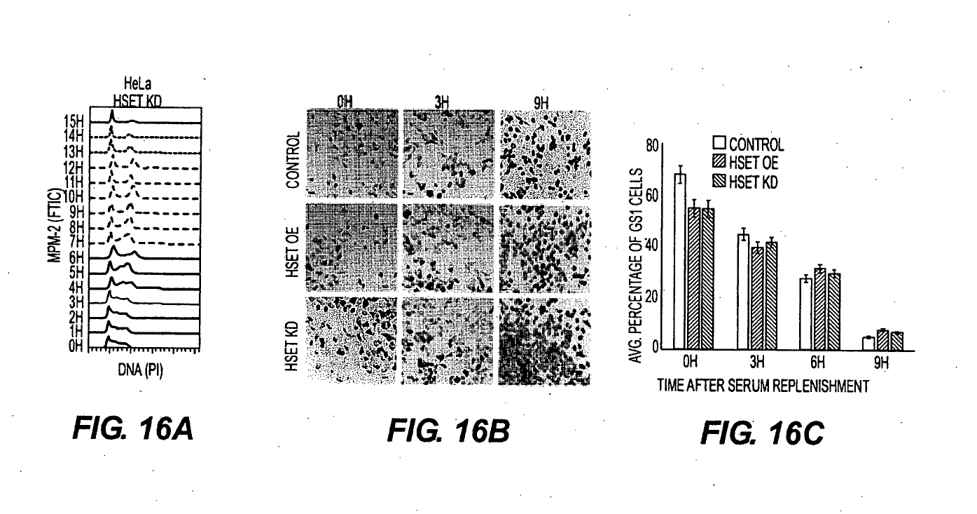

FIGS. 16A-16C show cell cycle kinetics in HeLa cells upon HEST OE and KD. FIG. 16A shows FACS profiles representing DNA content profiles at various time-points (indicated on y-axis) after release of synchronized HeLa-HSET-KD cells from the point of thymidine block (0 h) to the point after mitotic exit (15 h). Green lines represent S phase, red lines represent G2 phase and blue lines represent M phase. FIG. 16B depicts micrographs showing HeLa cells transfected with a control vector (CV), HSET overexpression (OE) contruct or an HSET knockdown (KD) construct expressing HSET siRNA in different phases of cell cycle when released from serum starvation by using a Cell-Clock assay kit. Yellow color depicts G1 phase cells, yellowish-green color depicts S phase, light blue color depicts G2 phase and dark blue color depicts M phase. FIG. 16C depicts bar graphs representing average percentage of cells in G1 phase out of total cells counted in 5 random fields, from 0 h to 9 h after serum replenishment (p<0.005).

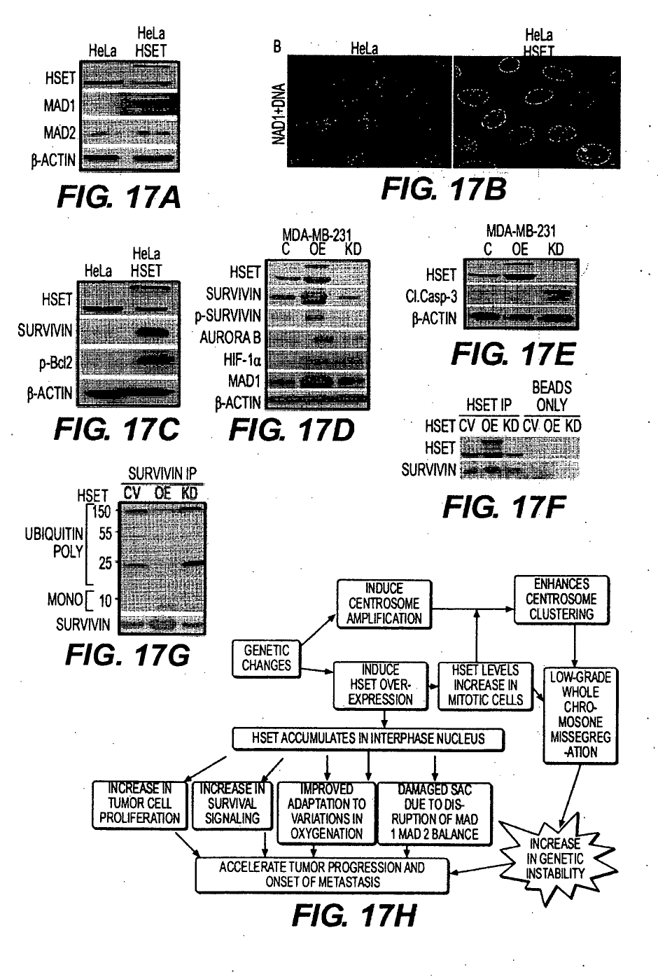

FIGS. 17A-17G show HEST overexpression upregulates survival proteins and disrupts balance of checkpoint proteins. FIG. 17A depicts immunoblots showing HSET, Mad1 and Mad2 protein levels in HeLa and HeLa-HSET-GFP cells. β-actin was used as a loading control for all Western blots. FIG. 17B depicts immunofluorescence micrographs showing Mad1 (green) levels and localization in HeLa and HeLa-HSET-GFP cells. FIG. 17C depicts immunoblots showing the expression levels of survival proteins (survivin, p-Bcl2) in HeLa and HeLa-HSET-GFP cells. FIG. 17D depicts immunoblots showing the expression of proteins associated with cell survival, cell cycle regulation, spindle assembly checkpoint and adaptation to hypoxia in MDA-MB-231 cells transiently transfected with a control GFP vector (C) as compared with MDA-MB-231 cells transiently transfected with HSET-pEGFP plasmid (OE) or an HSET-siRNA plasmid (KD). FIG. 17E depicts immunoblots showing HSET and cleaved caspase-3 protein expression in MDA-MB-231 cells transiently transfected with control vector (CV), HSET pEGFP plasmid (OE) or HSET siRNA (KD), followed by UV-C exposure at 25 J/m² for 10 min. FIG. 17F depicts immunoblots showing HSET and survivin protein levels in MDA-MB-231 transfected with control vector (CV), HSET overexpression (OE) plasmid or HSET knockdown (KD) plasmid wherein HSET was immunoprecipitated (HSET IP) or not immunoprecipitated (beads only) followed by immunoblotting against survivin. FIG. 17G depicts immunoblots of survivin complexes immunoprecipitated from MDA-MB-231 cells (transfected with control, overexpression, or knockdown vectors) and immunoblotted for survivin and ubiquitin. FIG. 17H is a schematic model depicting the involvement of HSET in tumor progression and metastasis via previously established mitotic pathways (green boxes) and interphase-specific pathways suggested by the present data (blue boxes). The dotted arrow indicates an unknown and indirect modulation of various downstream pathways by overexpressed nuclear HSET. C=control GFP vector.

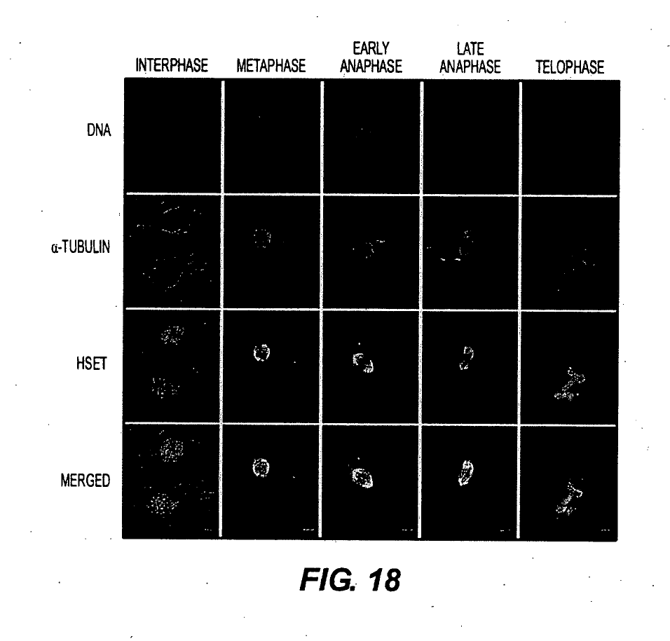

FIG. 18 depicts confocal micrographs showing HSET localization in various phases of cell cycle. HeLa cells were co-immunostained with HSET (green) and α-tubulin (red) antibodies. DNA was stained with DAPI (blue). Nuclear localization of HSET is clearly visible in interphase and telophase, whereas it is seen to be localized on minus-ends of microtubules in mitotic spindles during metaphase and anaphase. Scale bar 5 μm.

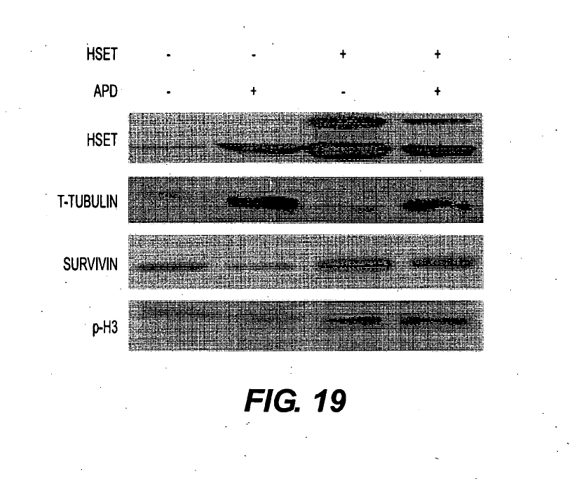

FIG. 19 depicts proliferation and survival effects of HSET overexpression in HeLa cells with or without amplified centrosomes by immunoblot analysis. Depending on the conditions, centrosome amplification (indicated by accumulation of centrosomal γ-tubulin), upregulated survival signaling (indicated by increased survivin levels) and proliferation (increase in p-H3 levels) to varying extents. APD: apidicolin.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the broadest possible scope consistent with the principles and features disclosed herein.

Unless defined otherwise, all technical and scientific terms used herein have the same meanings as commonly understood by one of skill in the art to which the disclosed method and compositions belong. It must be noted that as used herein and in the appended claims, the singular forms “a,” “an,” and “the” include plural reference unless the context clearly dictates otherwise. Thus, for example, reference to “small molecule drug” includes a plurality of such small molecule drugs, reference to “the small molecule drug” is a reference to one or more small molecule drugs, including equivalents thereof known to those skilled in the art, and so forth.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, the term “triple negative breast cancer” (TNBC) refers to breast cancers in which the tumor cells score negative (i.e., using conventional histopathology methods) for estrogen receptor (ER) and progesterone receptor (PR), both of which are nuclear receptors (i.e., they are predominantly located at cell nuclei), and are not amplified for epidermal growth factor receptor type 2 (HER2 or ErbB2), a receptor normally located on the cell surface. The tumor cells should be considered negative for expression of ER and PR if less than 5% of the tumor cell nuclei are stained for ER and PR expression using standard immunohistochemical techniques. Tumor cells are considered highly amplified for HER2 if, when tested with a HercepTest™Kit (Code K5204, Dako North America, Inc., Carpinteria, Calif.), a semi-quantitative immunohistochemical assay using a polyclonal anti-HER2 primary antibody, they yield a test result score of 3+, or, they test HER2 positive by fluorescence in-situ hybridization (FISH). As used herein, tumor cells are considered negative for HER2 overexpression if they yield a test result score of 0 or 1+, or 2+, or if they are HER2 FISH negative.

The term “patient” includes a human or other mammalian animal that receives either prophylactic or therapeutic treatment.

The term “gene product,” refers to the transcription product of a gene, such as mRNA, and the translation product of a gene, such as protein.

The term “therapeutic agent” includes any substance, molecule, element, compound, entity, or a combination thereof having a therapeutic effect in a triple negative breast cancer patient. It includes, but is not limited to, e.g., proteins, oligopeptides, small organic molecules, polysaccharides, polynucleotides, and the like. A therapeutic agent can be a natural product, a synthetic compound, a chemical compound or a combination of two or more substances.

The term “inhibitor of HSET” means any agent or compound that reduces, or decreases, or lessens the expression or activity of HSET kinesin, wherein the term “expression” should be understood to mean expression of HSET mRNA or expression of HSET protein in a cell and wherein the term “activity” should be understood to mean the enzymatic activity or associated biological properties of HSET, including, but not limited to, ATPase activity and microtubule binding activity.

The term “an effective amount” refers to an amount of a therapeutic agent sufficient to effect treatment in a patient with triple negative breast cancer. In this context, “treating” should be understood to mean encompass treatment resulting in a decrease in tumor size; a decrease in rate of tumor growth; stasis of tumor size; inhibition of tumor metastases formation; a decrease in the number of metastases; improved progression-free survival (PFS) (e.g., calculated as the number of days from diagnosis to the first local recurrence or metastasis if one occurred); improved overall survival (OS) (e.g., calculated based on the number of days from diagnosis to death or last follow-up if death was not recorded); a decrease in invasiveness of the cancer; a decrease in the rate of progression of the tumor from one stage to the next; inhibition of tumor growth in a triple negative patient; regression of established tumors; decrease in the angiogenesis induced by the cancer; inhibition of growth and proliferation of cancer cells; and combinations thereof.

One aspect of the present application relates to a method of assessing the prognosis of a patient diagnosed with cancer, the method comprises the steps of (a) performing an assay on a biological sample comprising cancer cells from the patient to determine whether the cancer cells express an elevated level of nuclear HSET; and providing an assessment of the prognosis of the patient based on the result of step (a), wherein an elevated level of nuclear HSET in the cancer cells indicates a poorer prognosis. Specifically, high levels of nuclear HSET expression indicate a poor prognosis and poor overall survival, particularly without appropriate and aggressive treatment. In some embodiments, the cancer is breast cancer. In other embodiments, the cancer is triple negative breast cancer. In other embodiments, the cancer is ovarian cancer. In yet other embodiments, the cancer is colon cancer, head and neck cancer, bladder cancer and glioma. In other embodiments, the cancer is vaginal cancer, cervical cancer, uterine cancer, prostate cancer, anal cancer, stomach cancer, pancreatic cancer, insulinoma, adenocarcinoma, adenosquamous carcinoma, neuroendocrine tumor, lung cancer, esophageal cancer, oral cancer, brain cancer, medulloblastoma, neuroectodermal tumor, pituitary cancer, or bone cancer.

Although the patient can undergo tests to determine the stage or grade of their cancer, the present methods can provide a prognosis, allow for a more accurate prediction of outcome, and inform the treatment regime in the absence of staging or grading. The methods can be repeated at intervals throughout a course of treatment (e.g., at the beginning and end of a treatment regime or about every 4-6 months) as an indicator of the patient's responsiveness to a treatment. Thus, the methods are also useful in modifying a prognosis or updating an expected outcome over time.

Patients amenable to the prognostic and therapeutic methods described herein are patients who have been diagnosed as having breast cancer, which is determined to be triple negative. In one embodiment, the method includes identifying the patient as a person of African descent, such as an African American. As further demonstrated in the Examples below, nuclear HSET expression was significantly associated with the proliferation marker Ki67; clinicopathological factors (e.g., tumor grade, tumor stage, and tumor size); with the Nottingham prognostic index (NPI); and with triple negative status. Its expression was also highly associated with race, with African American women being 1.6 times as likely to present with nuclear localization compared to European American women, after adjusting for triple negative status. In multivariate analysis, increased nuclear HSET expression was associated with worse overall, progression-free, and metastasis-free survival (HR=1.37, 1.30, and 1.34, respectively, with p<0.05 for all). Within the African American subset, increased expression of nuclear HSET was associated with even worse overall survival (HR=1.56, p=0.006), progression-free survival (HR=1.44, p=0.012), and metastasis-free survival (HR=1.44, p=0.015) in multivariate analysis. Intriguingly, survival outcomes were significantly associated with nuclear but not cytoplasmic HSET.

A sample from a triple negative breast cancer patient can be obtained from breast cancer cells within the patient (e.g., a tumor) or a fluid sample therefrom. The cells can be obtained by a variety of methods. For example, the sample can be obtained by any procedure in which tumor cells are dislodged from the tumor (e.g., the tumor cells may be obtained from a tumor biopsy removed during a mastectomy, from an aspirate of the tumor, from a lavage or other procedure in which tumor cells are dissociated from the tumor, or from a portion of the tumor that has been surgically removed). In the event breast cancer cells break free from the tumor and circulate, they can be detected in a fluid sample from the patient (e.g., blood, serum, or plasma).

Most samples will utilize at least a dozen cells, and likely at least a few hundred cells (e.g., about 200-500 cells) or more. Once obtained, the sample may be treated according to the requirements of the impending test. For example, tissue to be analyzed by immunohistochemistry can be fixed and embedded for sectioning. Alternatively, whole cell extracts, nuclear extracts or fractions thereof can be processed from the tissues or cells for expression analysis by conventional techniques.

The step of determining whether a given patient's cells express an elevated level of nuclear HSET can be carried out by an immunohistochemical analysis of the sample or an analysis of a nuclear fraction or extract from the sample. For the immunohistochemical analysis, the sample can be directly exposed to a binding agent (e.g., an antibody such as a rabbit polyclonal anti-HSET antibody for a time and under conditions sufficient to allow the binding agent to specifically bind nuclear HSET. Alternatively, a nuclear extract of the sample may be prepared and analyzed for binding of nuclear HSET to the binding agent.

HSET binding agents and those for binding other co-regulated proteins can be prepared using methods known in the art. For example, an intact protein (i.e., full length HSET or a co-regulated protein) or an antigenic fragment thereof can be injected into a laboratory animal (such as a rodent or rabbit), from which antibody-containing blood is later collected. The antibodies generated can be further developed to generate, for example, monoclonal, chimeric, single chain and humanized antibodies, as well as biologically active fragments thereof (e.g., an Fab fragment) may prepared from any suitable immunoglobulin class (e.g., an IgG) according to established methodologies known in the art.

HSET binding antibodies useful in the present methods may be directed to any suitable epitope. For example, HSET binding antibodies may target the N-terminus of HSET (e.g., an epitope constituting residues 1-304; residues 1-152; or residues 151-218) or they may target the C-terminal region (e.g., residues 625-673; FIG. 1).

In addition to analyzing HSET expression, the present methods can include a step of determining whether the cells express other products (e.g., proteins or RNAs) that are upregulated with HSET. Exemplary products include Npap60L, cellular apoptosis susceptibility protein (CAS), protein regulator of cytokinesis 1(Prc1), Ki67, survivin, phospho-survivin, HIF-1-alpha, aurora kinase B, Mad1, p-Bcl2 FoxM1, Plk 1, Auror A and KPNA2. Any combination of these markers may be evaluated to determine whether their expression levels are elevated relative to normal breast tissue controls.

Npap60 is a nucleoporin that binds directly to importin α. In humans, there are two Npap60 isoforms: the long (Npap60L) and short (Npap60S) forms. Whereas Npap60S stabilizes the binding of importin α to classical nuclear localization signal (NLS)-cargo and suppresses nuclear import of NLS-cargo, Npap60L promotes the release of NLS-cargo from importin α and accelerates the nuclear import of NLS-cargo. Cellular apoptosis susceptibility protein (CAS), also known as exportin 2 promotes the dissociation of the Npap60/importin α complex. It is believed that regulation of nucleoporin complexation and dissociation plays a role in determining nuclear expression levels of HSET, as well prognosis in AA TNBC patients.

In a specific embodiment, the method further comprises the step of determining expression levels of Npap60L and CAS from the patient's biological samples and determining an Npap60L to CAS expression level ratio, wherein a ratio of <0.7 indicates a poorer prognosis for the patient compared to a patient with triple negative breast cancer with an Npap60L to CAS expression level ratio of >0.7.

In another embodiment, the method further comprises the step of performing an assay from the patient's breast cancer cells to determine whether the breast cancer cells express an elevated level of Prc1, FoxM1, plk1, KPNA2 and/or Aurora A, wherein an elevated level of nuclear HSET and Prc1, FoxM1, plk1, KPNA2 and/or Aurora A indicates a poorer prognosis for the patient compared to a patient with triple negative breast cancer expressing lower levels of nuclear HSET and Prc1, FoxM1, plk1, KPNA2 and/or Aurora A. In some embodiments, the breast cancer cells expressing elevated lavels of nuclear HSET and nuclear Prc1, FoxM1, plk1, KPNA2 and/or Aurora A indicates a poorer prognosis. Prc1 is a non-motor-microtubule-associated protein that appears to be co-regulated and co-localized with HSET.

Alternatively, or in addition, a TNBC patient's samples may be evaluated to determine whether the patient's breast cancer cells exhibit increased Cdk1 activity and/or increased levels of phosphorylated histone-H3 relative to normal breast tissue controls.

In certain embodiments, rather than testing for nuclear HSET expression, expression levels of HSET mRNAs and other co-regulated gene products are determined by RT-PCR as a prognostic gene expression signature in patients with triple negative breast cancer.

Expression levels, including percent increases in expression level over controls, may be determined at the protein level (e.g., by immunohistochemistry, Western blot, antibody microarray, ELISA, etc.) or at the mRNA level (e.g., by RT-PCR, QT-PCR, oligonucleotide array, etc.). Preferred methodologies for determining protein expression levels (and ratios therefrom) include the use of immunohistochemistry, ELISAs, antibody microarrays and combinations thereof. Preferred methodologies for determining mRNA expression levels (and ratios therefrom) include quantitative reverse transcriptase PCR (QT-PCR), quantitative real-time RT-PCR, oligonucleotide microarrays and combinations thereof.

Elevated expression levels of HSET proteins, HSET mRNAs and/or co-regulated proteins or mRNAs may represent increase(s) of 10%, 20%, 30%, 40%, 50%, 60%, 70%, 80%, 90% or 100% relative to normal breast tissue controls. In other embodiments, elevated expression levels may represent increase(s) of 2-fold, 3-fold, 5-fold, 10-fold, 20-fold, 50-fold or 100-fold increases relative to normal breast tissue controls. Similarly, increased Cdk1 activity and/or increased levels of phosphorylated histone-H3 may represent increase(s) of 10%, 20%, 30%, 40%, 50%, 60%, 70%, 80%, 90%, 100% (activity or phosphorylation) relative to normal breast tissue controls or may represent increase(s) of 2-fold, 3-fold, 5-fold, 10-fold, 20-fold, 50-fold or 100-fold increases relative to normal breast tissue controls.

In certain embodiments, ancestry analysis may be performed by SNP analysis using ancestry informative markers (AIMs) to identify a patient's geographic origin(s). AIM markers can reveal the geographic origin of regions of a genome in, for example, about 1 million by region size chunks. Reference genomes are available for each geographic region to which samples are compared to identify the geographic origin(s) based on markers present in the patient's genome from up to at least 500 years ago (before much of the recent intercontinental travel) and can be used to identify those of African descent. In certain embodiments, ancestry analysis can be carried out commercially (e.g., 23andme and family tree DNA analysis companies).

### Administration of Therapeutic Agents

High levels of nuclear HSET expression indicate a poor prognosis and poor overall survival, particularly without appropriate and aggressive treatment. Accordingly, where the cells from a triple negative breast cancer patient is found to express an elevated level of nuclear HSET or total HSET mRNA, the patient may be further treated with one or more therapeutic agents.

In one embodiment, the patient is administered an inhibitor of HSET. The inhibitor of HSET can be a small molecule drug or a nucleic acid-based therapeutic, such as an siRNA, an shRNA-encoded expression vector or an antisense oligonucleotide, whereby the inhibitor inhibits the activity and/or expression of HSET in the targeted cell. Alternatively, or in addition, the patient may be administered an inhibitor of a protein that is upregulated with HSET. HSET co-regulated product targets include, but are not limited to Npap60L, CAS, Prc1, Ki67, survivin, phospho-survivin, HIF-1-alpha, aurora kinase B, p-Bcl2, Mad1, Plk1, FoxM1, KPNA2, Aurora A and combinations thereof. In other embodiments, the patient is administered one or more agents that block the nuclear accumulation of HSET during interphase.

siRNAs are double-stranded RNAs that can be engineered to induce sequence-specific post-transcriptional gene silencing of mRNAs. Synthetically produced siRNAs structurally mimic the types of siRNAs normally processed in cells by the enzyme Dicer. siRNAs may be administered directly in their double-stranded form or they may be expressed from an expression vector is engineered to transcribe a short double-stranded hairpin-like RNA (shRNA) that is processed into a targeted siRNA inside the cell. Suitable expression vectors include viral vectors, plasmid vectors and the like and may be delivered to cells using two primary delivery schemes: viral-based delivery systems using viral vectors and non-viral based delivery systems using, for example, plasmid vectors. Exemplary viral vectors may include or be derived from an adenovirus, adeno-associated virus, herpesvirus, retrovirus, vaccinia virus, poliovirus, poxvirus, HIV virus, lentivirus, retrovirus, Sindbis and other RNA viruses and the like.

As used herein, the term “oligonucleotide” refers to a single stranded nucleic acid containing between about 15 to about 100 nucleotides. An antisense oligonucleotide comprises comprise a DNA backbone, RNA backbone, or chemical derivative thereof, which is designed to bind via complementary binding to an mRNA sense strand of a target gene (such as HSET) so as to promote RNase H activity, thereby leading to degradation of the mRNA. Preferably, the antisense oligonucleotide is chemically or structurally modified to promote nuclease stability and/or increased binding. The single stranded antisense oligonucleotide may be synthetically produced or it may be expressed from a suitable expression vector. In addition, the antisense oligonucleotide may be modified with nonconventional chemical or backbone additions or substitutions, including but not limited to peptide nucleic acids (PNAs), locked nucleic acids (LNAs), morpholino backboned nucleic acids, methylphosphonates, duplex stabilizing stilbene or pyrenyl caps, phosphorothioates, phosphoroamidates, phosphotriesters, and the like.

In certain embodiments, the small molecule drug targets the motor domain of HSET and/or specifically binds to the HSET/microtubule binary complex so as to inhibit HSET's microtubule-stimulated and/or microtubule-independent ATPase activities. In a specific embodiment, the small molecule drug is AZ82 (shown below) or a therapeutically effective derivative, salt, enantiomer, or analog thereof.

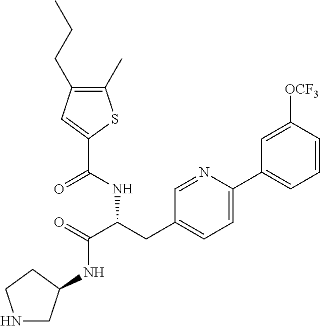 [SMILES: CCCc1cc(C(=O)N[C@H](Cc2ccc(-c3cccc(OC(F)(F)F)c3)nc2)C(=O)N[C@@H]2CCNC2)sc1C]

AZ82 binds specifically to the KIFC1/microtubule (MT) binary complex and inhibits the MT-stimulated KIFC1 enzymatic activity in an ATP-competitive and MT-noncompetitive manner with a Ki of 0.043 μM. Treatment with AZ82 causes centrosome declustering in BT-549 breast cancer cells with amplified centrosomes.

Other small molecule HSET antagonists and/or centrosome declustering agents include, but are not limited to griseofulvin; noscapine, noscapine derivatives, such as brominated noscapine (e.g., 9-bromonoscapine), reduced bromonoscapine (RBN), N-(3-brormobenzyl) noscapine, aminonoscapine and water-soluble derivatives thereof; CW069; the phenanthridene-derived poly(ADP-ribose) polymerase inhibitor, PJ-34; N2-(3-pyridylmethyl)-5-nitro-2-furamide, N2-(2-thienylmethyl)-5-nitro-2-furamide, N2-benzyl-5-nitro-2-furamide, an anthracine compound as described in U.S. Patent Application Publication 2008/0051463; a 5-nitrofuran-2-carboxamide derivative as described in U.S. Provisional Application 61/619,780; and derivatives and analogs therefrom.

In certain embodiments, the patient may be additionally administered a poly(ADP-ribose) polymerase (PARP) inhibitor, an inhibitor of the Ras/MAPK pathway, an inhibitor of the PI3K/AKT/mTOR pathway, an inhibitor of FoxM1, Hif1α, surviving, Aurora, Plk1 or a combination thereof. Exemplary PARP inhibitors include, but are not limited to olaparib, iniparib, velaparib, BMN-673, BSI-201, AG014699, ABT-888, GPI21016, MK4827, INO-1001, CEP-9722, PJ-34, Tiq-A, Phen, PF-01367338 and combinations thereof. Exemplary Ras/MAPK pathway agents include, but are not limited to MAP/ERK kinase (MEK) inhibitors, such as trametinib, selumetinib, cobimetinib, CI-1040, PD0325901, AS703026, R04987655, R05068760, AZD6244, GSK1120212, TAK-733, U0126, MEK162, GDC-0973 and combinations thereof. Exemplary PI3K/AKT/mTOR pathway inhibitors include, but are not limited to everolimus, temsirolimus, GSK2126458, BEZ235, PIK90, PI103 and combinations thereof.

### Other Prescribed Therapies

Alternatively, or in addition to administering an HSET-targeted therapeutic, a patient expressing high levels of nuclear HSET may be additionally treated with adjuvant chemotherapeutic agents to further reduce the risk of adverse events, such as metastasis, disease relapse, and poor survival. Adjuvant chemotherapies may include administration of cyclophosphamide, taxanes, such as docetaxel and paclitaxel; anthracyclines, such as epirubicin and doxorubicin; gemcitabine, cisplatin, fluorouracil, ixabepilone, capecitabine, epidermal growth factor receptor-targeting agents, and combinations thereof.

The appropriate dosage (“therapeutically effective amount”) of the therapeutic agent(s) will depend, for example, on the severity and course of the breast cancer, the mode of administration, the bioavailability of the therapeutic agent(s), previous therap(ies), the age and weight of the patient, the patient's clinical history and response to the therapeutic agent(s), the type of the therapeutic agent used, discretion of the attending physician, etc. The therapeutic agent(s) are suitably administered to the patent at one time or over a series of treatments and may be administered to the patient at any time from diagnosis onwards. The therapeutic agent(s) may be administered as the sole treatment or in combination with other drugs or therapies useful in treating the breast cancer. When used with other drugs, the therapeutic agent(s) may be used at a lower dose to reduce toxicities and/or side effects.

The therapeutic agent(s) may be administered to the patient with known methods, such as intravenous administration as a bolus or by continuous infusion over a period of time, by intramuscular, intraperitoneal, intracerebrospinal, subcutaneous, intra-articular, intrasynovial, intrathecal, oral, topical and/or inhalation routes. As a general proposition, the therapeutically effective amount(s) of the above described therapeutic agent(s) will be in the range of about 1 ng/kg body weight/day to about 100 mg/kg body weight/day whether by one or more administrations. In a particular embodiments, each therapeutic agent is administered in the range of from about 1 ng/kg body weight/day to about 10 mg/kg body weight/day, about 1 ng/kg body weight/day to about 1 mg/kg body weight/day, about 1 ng/kg body weight/day to about 100 μg/kg body weight/day, about 1 ng/kg body weight/day to about 10 μg/kg body weight/day, about 1 ng/kg body weight/day to about 1 μg/kg body weight/day, about 1 ng/kg body weight/day to about 100 ng/kg body weight/day, about 1 ng/kg body weight/day to about 10 ng/kg body weight/day, about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, about 1 μg/kg body weight/day to about 10 mg/kg body weight/day, about 1 μg/kg body weight/day to about 1 mg/kg body weight/day, about 1 μg/kg body weight/day to about 100 μg/kg body weight/day, about 1 μg/kg body weight/day to about 10 μg/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In certain embodiments, the therapeutic agent(s) are administered at a dose of 500 μg to 20 g every three days, or 10 μg to 400 mg/kg body weight every three days. In other embodiments, each therapeutic agent is administered in the range of about 10 ng to about 100 ng per individual administration, about 10 ng to about 1 μg per individual administration, about 10 ng to about 10 μg per individual administration, about 10 ng to about 100 μg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 μg per individual administration, about 100 ng to about 10 μg per individual administration, about 100 ng to about 100 μg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 μg to about 10 μg per individual administration, about 1 μg to about 100 μs per individual administration, about 1 μg to about 1 mg per individual administration, about 1 μg to about 10 mg per individual administration, about 1 μg to about 100 mg per individual administration, about 1 μg to about 1000 mg per injection, about 1 μg to about 10,000 mg per individual administration, about 10 μg to about 100 μs per individual administration, about 10 μg to about 1 mg per individual administration, about 10 μg to about 10 mg per individual administration, about 10 μg to about 100 mg per individual administration, about 10 μg to about 1000 mg per injection, about 10 μg to about 10,000 mg per individual administration, about 100 μg to about 1 mg per individual administration, about 100 μg to about 10 mg per individual administration, about 100 μg to about 100 mg per individual administration, about 100 μg to about 1000 mg per injection, about 100 μg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The therapeutic agent(s) may be administered daily, or every 2, 3, 4, 5, 6 and 7 days, or every 1, 2, 3 or 4 weeks.

In other particular embodiments, the therapeutic agent(s) are administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day. As expected, the dosage(s) will be dependent on the condition, size, age and condition of the patient.

Another aspect of the present application relates to a method for treating TNBC patients with high nuclear HSET accumulation by increasing the Npap60L-to-Npap60S ratio in these patients. In some embodiments, the method comprises the step of administering to a TNBC patient with high nuclear HSET accumulation an effective amount of an agent that increases the Npap60L-to-Npap60S ratio in the breast tissue of the patient.

Another aspect of the present application relates to a method for treating TNBC patients with high nuclear HSET accumulation by inhibiting the expression or activity of Prc1 in these patients. In some embodiments, the method comprises the step of administering to a TNBC patient with high nuclear HSET accumulation an effective amount of an agent that inhibits the expression or activity of Prc1 in the breast tissue of the patient.

Another aspect of the present application relates to a method for treating TNBC patients with high nuclear HSET accumulation by inhibiting the expression or activity of FoxM1 and/or Plk1 in these patients. In some embodiments, the method comprises the step of administering to a TNBC patient with high nuclear HSET accumulation an effective amount of an agent that inhibits the expression or activity of FoxM1 and/or Plk1 in the breast tissue of the patient.

Another aspect of the present application relates to a method for treating TNBC patients with high nuclear HSET accumulation by inhibiting the expression or activity of Aurora A and/or KPNA2 in these patients. In some embodiments, the method comprises the step of administering to a TNBC patient with high nuclear HSET accumulation an effective amount of an agent that inhibits the expression or activity of Aurora A and/or KPNA2 in the breast tissue of the patient.

Another aspect of the present application relates to a kit for determining elevated expression of HSET. In some embodiments, the kit includes an HSET binding agent along with one or more secondary binding agents specifically binding to one or more gene product(s) upregulated before, during or after (e.g., subsequent to and as a result of) HSET elevation. In some embodiments, the HSET binding agent and/or the one or more secondary binding agents are antibodies. In some embodiments, the one or more gene product(s) are selected from the group consisting of gene products of Npap60L, CAS, Prc1, Ki67, survivin, phospho-survivin, HIF1α, aurora kinase B, Mad1, p-Bcl2, FoxM1, Plk1, Aurora A and KPNA2. In some embodiments, the kit further includes one or more reagents for preparation of a nuclear fraction or extract. In some embodiments, the kit further includes one or more reagents for immunohistochemistry. In some embodiments, the one or more reagents for immunohistochemistry include reagents for staining the nuclei. In some embodiments, the kit further includes instructions for using the reagents for the detection of HSET and/or the one or more gene products.

### EXAMPLES

### Materials and Methods

### Study Population and Tumor Tissue Samples

Approval from the Emory Institutional Review Board (IRB) was obtained for all aspects of these studies. Archival paraffin-embedded tissue samples were collected during patient care and diagnostics. Since no direct patient interaction occurred, a formal consent was not required for testing of these samples. Surgical pathology files from Emory University and Grady Memorial Hospitals (Atlanta, Ga.) between the years 2003-2008 were searched for African American and European American breast carcinoma samples with clinicopathological, demographic, and outcome information (e.g., tumor grade, stage, and size; ER, PR, and HER2 status; Ki67 staining; age; ethnicity; overall, progression-free, and metastasis-free survival).

Samples from 193 breast carcinoma patients were obtained, the characteristics of which are provided in Table 1:

TABLE 1

| Variable | Level | N % | |
|---|---|---|---|
| Race | EA | 44 | (22.8) |
| | AA | 149 | (77.2) |
| TN Status | No | 60 | (31.1) |
| | Yes | 133 | (68.9) |
| Tumor Size | ≦2 | 89 | (46.4) |
| | ≧2 | 103 | (53.6) |
| | Missing | 1 | |
| Grade | 1 | 21 | (11.0) |
| | 2 | 62 | (32.5) |
| | 3 | 108 | (56.5) |
| | Missing | 2 | |
| Stage | I/II | 120 | (63.8) |
| | III/IV | 68 | (36.2) |
| | Missing | 5 | |
| Positive LN | Absent | 112 | (60.5) |
| | Present | 73 | (39.5) |
| | Missing | 8 | |
| Variable | Statistic | | |
| HSET Nucleus WI | Mean (Std Dev) | 63.91 | (53.45) |
| | Median (Min-Max) | 45 | (0-240) |
| | Missing | 4 | |
| HSET Cytoplasm WI | Mean (Std Dev) | 126 | (90.80) |
| | Median (Min-Max) | 100 | (0-300) |
| | Missing | 2 | |
| HSET Total WI | Mean (Std Dev) | 189 | (118) |
| | Median (Min-Max) | 180 | (0-480) |

| | |
|---|---|
| Missing | 6 |

### Immunohistochemistry and HSET Scoring

Tissue microarrays (TMAs) were constructed from cores (2 each, 1 mm in diameter) of breast tumors along with normal breast tissue (controls), all of which had been previously fixed with formalin and embedded in paraffin. Five micron sections were taken from the TMAs for immunohistochemistry. The TMAs were processed for immunostaining by performing antigen retrieval in citrate buffer (pH 6.0) in a pressure-cooker (15 psi) for 3 minutes. Immunostaining for HSET at a 1:1000 dilution was performed using a rabbit polyclonal antibody.

HSET staining intensity was assessed for both the cell nucleus and cytoplasm by an experienced pathologist who was blinded to patient and tissue characteristics. Nuclear and cytoplasmic staining were assessed semi-quantitatively by assigning a relative intensity score (0=none, 1=low, 2=moderate, or 3=high). The percentage of cell nuclei or cytoplasms demonstrating any HSET positivity (i.e., a score of 1, 2, or 3) was also determined. The average percentage was taken from the two cores that represented each sample and used for subsequent calculations. The product of the relative intensity and percent positivity was recorded as the weighted index (WI) for both the nucleus and cytoplasm. The sum of the nucleus WI and cytoplasm WI was recorded as the total WI. The HSET WI for the nucleus had an average of 63.91, median of 45, and a range of 0-240. The HSET WI for the cytoplasm was generally higher and had an average of 126.00, a median of 100, and a range of 0-300. These and other statistics regarding HSET staining can be found in Table 1.

### Statistical Methods

All statistical analyses were conducted using SAS Version 9.3 with p<0.05 considered statistically significant. Optimal cut points using maximum log-rank test statistic method for HSET WI with respect to survival outcomes were not found, which supported treating HSET WI as a continuous variable in the model. Furthermore, there is no published threshold for a hazardous level of HSET expression in breast cancer. Consequently, HSET was initially treated as a continuous variable. In subsequent analyses of African American patients only, optimal cut points for HSET nucleus WI with respect to survival outcomes were found; therefore it was categorized based on those cut points for the subgroup analysis.

Overall survival was defined as the number of days from diagnosis to death or last follow-up if death was not recorded. Progression-free survival was defined as the number of days from diagnosis to the first local recurrence, metastasis, or death, whichever occurred first, or the last follow-up if the patient did not experience an event. Metastasis-free survival was defined as the number of days from diagnosis to the first metastasis, or death, whichever occurred first, or the last follow-up if the patient did not experience an event. Covariates included TN status, tumor size, grade, stage, positive lymph nodes, age at diagnosis, Nottingham Prognostic Index (NPI) and Ki67 WI. NPI was calculated from grade, positive lymph nodes, and 0.2× tumor size.

Descriptive statistics were reported for all variables. The unadjusted association of all covariates with continuous HSET nucleus was assessed using ANOVA and the Kruskal-Wallis test for categorical covariates and Pearson and Spearman correlation coefficients for numerical covariates. Since the distribution of HSET nucleus WI was right skewed, it was square root transformed for the purpose of ANOVA. The association of race with HSET was additionally assessed adjusting for TN status. A general linear model was used to predict HSET.

The unadjusted association of each covariate with overall, progression-free, and metastasis-free survival was assessed using Cox proportional hazards models. Additionally, Cox models were fit including nuclear HSET WI. Main effects models were fit including race and HSET. Additionally, the covariates, TN status, stage, age, and NPI, were entered into the model subject to a backward variable selection method with an alpha=0.20 removal criteria. NPI was used in place of tumor size, grade, and positive lymph nodes. Ki67 was not included due to the high number of missing values. Subgroup analysis was also repeated on TN patients and African American (AA) patients. Among AA patients, TN status was forced into the models instead of race. Unadjusted Kaplan-Meier survival curves were produced for each outcome stratified by HSET group for African Americans. Survival differences between the groups were assessed using the log-rank test.

### In Silico Analysis of HSET Gene Expression

I. Data Collection:

One channel microarray data for various cancers were collected from Gene Expression Omnibus (GEO) database (Edgar R et al., Nucleic Acids Res., 2002, 30:207-210). The list of the GSE ID's is given in Table 2.

TABLE 2

| Cancer | Normal Samples GEO Series | Cancer Samples | N | N |
|---|---|---|---|---|
| Type | ID | GEO Series ID | Normal | Cancer |
| Glioblastoma | GSE10878 | GSE10878 | 3 | 20 |
| Lung Cancer | http://www.broadinstitute.org/mpr/publications/projects/ | | 17 | 19 |
| | Lung Cancer/ | | | |
| Leukemia | http://www.broadinstitute.org/mpr/publications/projects/ | | 16 | 6 |
| | Leukemia/ | | | |
| Breast | GSE10797 | GSE7390, GSE18864 | 16 | 179 |
| Cancer | | | | |
| Colon | GSE4107 | GSE18088 | 10 | 53 |
| Cancer | | | | |
| Cervical | GSE9750 | GSE9750 | 21 | 33 |
| Cancer | | | | |

II. Data Pre-Processing:

One channel microarray data was processed using Robust Multiarray (RMA) normalization (Gautier L et al., Bioinforatics, 2004, 20: 307-315) and was further used for gene expression analysis.

III. Analysis of HSET Gene Expression:

Log₂ n transformed HSET expression levels were analyzed in glioblastoma, leukemia, lung, breast, colon and cervical tumor samples as compared to their corresponding normal tissues.

### Clinical Tissue Samples:

All paraffin-embedded tissue slides were commercially obtained (from Accumax, and US Biomax). A subset of well-annotated tissue microarrays (TMAs) (193 biospecimens) with information on clinical outcomes, were obtained from Dr. Gabriela Oprea, Grady Memorial Hospital. The Emory Institutional Review Board (IRB) approval was obtained for all aspects of the study.

### Cell Culture and Transfection:

HeLa-HSET-GFP cells were generously provided by Claire Walczak (Indiana University). HeLa and HeLa-HSET-GFP, MDA-MB-231 cells were grown in DMEM supplemented with 10% FBS and 1% penicillin/streptomycin. Briefly, cells were seeded onto 100-mm plates 1 day prior to transfection. Plasmid DNA (5 μg) and 15 μl of DharmaFECT 4 transfection reagent (Thermo Scientific, PA, USA) were used for each transfection. HSET-pEGFP plasmid was generously provided by Claire Walczak. Cells overexpressing HSET were selected in the medium containing G418 (400 μg/ml). The G418-resistant colonies were collected and examined for HSET expression. SMARTpool: ON-TARGETplus KIFC1 siRNA (Dharmacon, PA, USA) was used to knockdown HSET in MDA-MB-231 cells.

### Cellular Protein Preparation, Western Blotting, Immunofluorescence and Antibodies:

Cells were cultured to ˜70% confluence and protein lysates were collected following transfection or otherwise. Fresh frozen tissue sections were first sonicated and lysates were then prepared. The immune-reactive bands corresponding to respective primary antibodies were visualized by the Pierce ECL chemiluminescence detection kit (Thermo Scientific). β-actin was used as loading control. For immunofluorescence staining, cells grown on glass coverslips were fixed with cold (−20° C.) methanol for 10 min and blocked by incubating with 2% bovine serum albumin/PBS.0.05% Triton X-100 at 37° C. for 1 h. Specific primary antibodies were incubated with coverslips for 1 h at 37° C. at the recommended dilution. The cells were washed with 2% bovine serum albumin/PBS for 10 min at room temperature before incubating with a 1:2000 dilution of Alexa 488- or 555-conjugated secondary antibodies. Cells were mounted with Prolong Gold antifade reagent that contains 4′,6-diamidino-2-phenylindole (DAPI) (Invitrogen). Polyclonal rabbit anti-HSET antibody was provided by Claire Walczak. Antibodies against α-tubulin and β-actin were from Sigma (St. Louis, Mo., USA). Antibodies against γ-tubulin, α-tubulin and ƒ1-actin were from Sigma (St. Louis, Mo., USA). Anti-Mad2 antibody was from BD Biosciences (Pharmingen, San Jose, Calif., USA). Antibodies against p-Bcl2 and cleaved caspase-3 were from Cell Signaling (Danvers, Mass., USA). Alexa 488- or 555-conjugated secondary antibodies were from Invitrogen (Carlsbad, Calif., USA). Anti-Mad1 antibody was a generous gift from Andrea Musacchio. Anti-Ki67 antibody was from Abcam (Cambridge, Mass., USA). Horseradish peroxidase-conjugated secondary antibodies were from Santa Cruz Biotechnology (Santa Cruz, Calif., USA).

### Kinase Activity Assay:

To examine cdk1 kinase activity, anti-cdk1 antibody was used to selectively immunoprecipitate cdk1-containing complexes from HeLa and HeLa-HSET-GFP cell lysates. The resulting immunoprecipitate was incubated with pure histone-H3 protein in the presence of p32-labelled ATP and kinase buffer. The kinase assay reaction allowed immunoprecipitated cdk1 to phosphorylate histone-H3 in vitro, the extent of which was measured by immunoblotting using phosphohistone-H3 antibody from Cell Signaling (MA, USA). Histone-H3 protein was from Millipore (MA, USA) and ATP was from Cell Signaling.

### Fluorescence In Situ Hybridization:

The slide samples from tumor cell lines or tumor tissue were hybridized by 2-color FISH with an HSET-specific BAC probe (RPCI-11 602P21, green) and a chromosome 6 centromere (CH514-7B4, red) (BACPAC). The HSET and centromere 6 probes were labeled with Cy3-dUTP (red) and FITC-dUTP (green), respectively, and hybridized with nuclei from cell lines or tumor tissue samples. Plasmids for production of a particular FISH probe were combined in equimolar amounts (55-70 pM). Nick translation was performed on 2 μg of this substrate by using Nick translation kit (Abbott Molecular, IL, USA). The translation product was denatured for 3 mins at 95° C. followed by fast cooling on ice and confirmed in 1.5% agarose gel electrophoresis as a smear of fragments ranging between 100 and 300 bp. A 2 min denaturation at 76° C. was followed by overnight (12-16 h) incubation at 37° C. Hybridization of the FISH probes was carried out in LSI/WCP hybridization buffer (Abbott Molecular, IL, USA). The slides were counterstained with DAPI (Invitrogen, NY, USA) and the Zeiss LSM 700 confocal microscope was used to capture FISH images. Results were expressed as a ratio of the number of copies of the HSET gene to the number of chromosome 6-centromeric markers.

### Flow Cytometry

Trypsinized cells were resuspended in PBS at 10⁶ cells/ml. Cells were then fixed by addition of ice-cold 70% ethanol. Ethanol-fixed cells were kept overnight at 4° C. before staining. Cells were pelleted and washed twice with PBS. Cell pellets were incubated for an hour at room temperature with mouse anti-MPM-2 antibody (Millipore, Mass., USA), followed by 1 h incubation with Alexa-488 anti-mouse secondary antibody (Life Technologies, NY, USA). Finally cells were washed, pelleted and resuspended in propidium iodide-containing isotonic buffer (0.1 mg/mL) and 0.5% Triton X-100. Cell cycle distribution was determined by flow cytometry using an LSR Fortessa Flow cytometer (BD Biosciences, CA, USA) and analyzed using Flowjo software (Tree Star, OR, USA).

### Trypan Blue Cell Exclusion Assay

Cells were cultured to ˜70% confluence followed by centrifuging and pellet was resuspended in 1 mL culture medium. 0.1 mL of 0.4% Trypan Blue solution was then added to 1 mL of cell suspension. The hemocytometer was loaded with 10 μL of the solution and examined immediately under a microscope. Live (white) and dead (blue) cells were counted and the percent cell viability was calculated using the following formula: percent viable cells=[1.00−(Number of live cells Number of total cells)]×100.

### BrdU Incorporation Assay

Asynchronous proliferating HeLa and HeLa-HSET-GFP cells were grown on coverslips to a confluency of ˜70% and then incorporated with 10 μM BrdU for 1 h followed by fixation with 70% ethanol at room temperature and immersion in 0.07 N NaOH for 2 minutes (which was then neutralized with PBS, pH 8.5). Coverslips were then incubated in 2% bovine serum albumin/PBS.0.05% Triton X-100 at 37° C. for 1 h followed by immunostaining using a 1:1000 dilution of Anti-BrdU-FITC antibody (BD Biosciences, San Jose, Calif., USA). BrdU positive cells, indicative of cell proliferation, were captured on a Zeiss Axioplan-2 fluorescence microscope (20×).

### Immunoprecipitation and Endogenous Ubiquitination Analysis

MDA-MB-231 cells were transiently transfected with CV, HSET-pEGFP plasmid or HSET SMARTpool siRNA as described above, and lysates were collected. Cell lysates were clarified by centrifugation at 10,000 rpm, and the supernatants (500 μg of protein) were subjected to immunoprecipitation with 4 μL of anti-HSET or anti-survivin antibodies. After overnight incubation at 4° C., protein A-agarose beads were added and left at 4° C. overnight. Immunocomplexes were then subjected to Western blot analysis as described previously. Western blot analysis with anti-ubiquitin antibody (Life Sensors, PA, 1:500) was performed by first incubating the PVDF membrane with 0.5% glutaraldehyde/PBS pH 7.0 for 20 min and then probing for the antibody.

### Cell-Clock Assay

HeLa cells were grown to 60-70% confluence and then transiently transfected with CV, HSET-pEGFP plasmid or HSET SMARTpool siRNA as described above. After 48 h, the cell clock dye (Biocolor, UK) (pre-warmed at 37° C.) was added (150 μl per well in 12-well plate) and the cells were incubated at 37° C. for 1 h. Dye was the washed twice with pre-warmed DMEM medium. Fresh medium was added and the cells were imaged in bright field (to assess different phases of cell cycle) and fluorescent (red for PI) channel. Cell clock dye is a redox dye, which is readily taken up by live cells. In G1 phase, the dye in its reduced form is yellow in color, while in the intermediate state it is green (S and G2 phase) before turning dark blue in the fully oxidized form (mitosis).

### Example 1

### HSET Expression and Breast Cancer Progression

To probe whether HSET may serve as a prognostic biomarker in breast cancer, the relationship between HSET expression and disease progression was investigated in a dataset of 193 breast cancer patients. Since HSET was treated as a continuous variable, hazard ratios (HR) for nuclear, cytoplasmic, and total HSET were calculated based on the respective standard deviations for these values (Table 1). HSET expression was evaluated using a WI (the product of the staining intensity and the proportion of positive cells). Both the expression level in the nucleus and cytoplasm, along with their sum, were assessed. In univariate analysis, nuclear HSET expression at the time of diagnosis was found to be significantly associated with worse progression- and metastasis-free survival (HR=1.23, p=0.046 and HR=1.27, p=0.025, respectively), with a borderline-significant trend noticed for overall survival (HR=1.25, p=0.052), as shown in Table 3:

TABLE 3
Univariate correlation of HSET WIs with overall, progression-free, and metastasis-
free survival (OS, PFS, and MFS, respectively). HR = hazard ratio, CI = confidence interval

| | OS | | PFS | | MFS | |
|---|---|---|---|---|---|---|
| HSET WI | HR (CI) | P | HR (CI) | P | HR (CI) | P |
| Nucleus | 1.25 (1.00, 1.57) | 0.052 | 1.23 (1.00, 1.51) | 0.046 | 1.27 (1.03, 1.57) | 0.026 |
| Cytoplasm | 1.00 (0.78, 1.29) | 0.99 | 0.99 (0.79, 1.24) | 0.92 | 0.98 (0.78, 1.24) | 0.87 |
| Total | 1.10 (0.86, 1.41) | 0.46 | 1.08 (0.85, 1.36) | 0.53 | 1.08 (0.85, 1.38) | 0.52 |

No significant or borderline-significant associations were found for cytoplasmic or total HSET expression with overall, progression-free, or metastasis-free survival (Table 2). African American race was also associated with worse overall survival (HR=2.66, p=0.023), although TN status was not (p=0.030).

Given the association of nuclear HSET with survival outcomes, additional prognostic indicators were examined for a further association with nuclear HSET expression. As the distribution of the nuclear HSET WI was right-skewed, it was square root transformed in order to perform ANOVA. Nuclear HSET was found to be significantly and positively associated with TN status, tumor size, tumor grade, and NPI (p<0.001 for all) along with tumor stage (p=0.013), as shown in Table 4.

TABLE 4
Association of nuclear HSET WI (square root transformed) with
demographic and clinicopathological characteristics. EA = European
American, AA = African American, LN = lymph nodes,
WI = weighted index, NPI = Nottingham Prognostic Index

| Variable | Level | N | Mean | P |
|---|---|---|---|---|
| Race | EA | 43 | 6.29 | 0.10 |
| | AA | 146 | 7.34 | |
| TN Status | No | 59 | 5.50 | <0.001 |
| | Yes | 130 | 7.82 | |
| Tumor Size (cm) | ≧2 | 87 | 6.01 | <0.001 |
| | <2 | 101 | 8.01 | |
| Grade | 1 | 21 | 4.23 | <0.001 |
| | 2 | 59 | 5.98 | |
| | 3 | 107 | 8.25 | |
| Stage | I/II | 118 | 6.66 | 0.014 |
| | III/IV | 66 | 8.05 | |

| Variable | N | Pearson CC | P |
|---|---|---|---|
| Age | 189 | −0.14 | 0.05 |
| NPI | 179 | 0.34 | <0.001 |
| Ki67 | 102 | 0.32 | <0.001 |

In univariate analysis of all patients (Table 3), nuclear HSET expression was elevated in African American (AA) patients as compared to European American (EA) patients, although this association did not reach statistical significance (p=0.1). Importantly, nuclear HSET was highly significantly associated with TN status, tumor size ≧2 cm, grade, NPI, and Ki67 WI (p<0.001) and pronouncedly associated with stage (p=0.014). Ki67 is utilized as a proliferation marker in breast cancer and may be associated with worse survival, although no significant associations between Ki67 and overall, progression-free, or metastasis-free survival were found by univariate analysis (p>0.40 for all).

### Example 2

### Nuclear HSET Expression is Associated with Worse Disease Progression in Multivariate Analysis

Having identified strong association of higher nuclear HSET with poorer prognostic indicators (such as TN status, tumor size, grade, NPI, and Ki67 WI and progression- and metastasis-free survival) by univariate analysis, multivariate analyses were carried out to evaluate whether the relationship between nuclear HSET expression and disease outcomes was retained after adjusting for standard prognostic indicators and possible confounding factors, such as NPI stage, age, and ethnicity. The associations of nuclear HSET with worse overall, progression-free, and metastasis-free survival retained significance and were in fact found to be stronger in multivariate analysis (HR=1.37, p=0.030; HR=1.30, p=0.044; and HR=1.34, p=0.035, respectively), as shown in Table 5:

TABLE 5
Multivariate correlation of HSET WIs with overall, progression-free,
and metastasis-free survival (OS, PFS, and MFS, respectively)
across the entire patient sample (with both African American (AA)
and European American (EA) patients). HR = hazard ratio,
CI = confidence interval.

| OS | | PFS | | MFS | |
|---|---|---|---|---|---|
| HR (CI) | P | HR (CI) | P | HR (CI) | P |
| 1.37 | 0.030 | 1.30 | 0.044 | 1.34 | 0.035 |
| (1.03, 1.82) | | (1.01, 1.69) | | (1.02, 1.75) | |

This finding confirms that nuclear HSET is not merely associated with these standard negative prognostic indicators such as Ki67, but is rather independently associated with worse outcomes. In the overall survival multivariate model, the hazard for AA patients was significantly greater than for EA patients (HR=2.95, p=0.031).

### Example 3

### The Univariate Relationship of HSET with Ethnicity is Restricted to TN Patients

Interrelationships between AA ethnicity, disease progression and mortality, and HSET expression were examined. Although AA patients are more likely to be diagnosed with TN receptor status, this does not appear to translate into worse clinical outcomes. Thus interrelationships between TN status, ethnicity, and HSET expression were analyzed. Within non-TN patients, there was no difference in HSET expression (nuclear, cytoplasmic, or total) between African American and European American patients (p>0.40 for all), as shown in Table 6:

TABLE 6
Association of HSET weighted indices (Wis) with race by Triple Negative
(TN) status. Sqrt = square root, European American (EA) patients,
AA = African American

| TN Status | Covariate | Statistic | EA | AA | P |
|---|---|---|---|---|---|
| No | Sqrt HSET Nucleus WI | N | 7 | 52 | 0.90 |
| | | Mean | 5.38 | 5.52 | |
| | HSET Cytoplasm WI | N | 7 | 51 | 0.86 |
| | | Mean | 84.29 | 79.12 | |
| | HSET Total WI | N | 7 | 50 | 0.96 |
| | | Mean | 114.86 | 116.44 | |
| Yes | Sqrt HSET Nucleus WI | N | 36 | 94 | 0.011 |
| | | Mean | 6.47 | 8.34 | |
| | HSET Cytoplasm WI | N | 37 | 96 | 0.023 |
| | | Mean | 174.32 | 134.69 | |
| | HSET Total WI | N | 36 | 94 | 0.369 |
| | | Mean | 236.19 | 215.48 | |

However, within TN patients, African American women demonstrated higher nuclear HSET expression (square-root transformed data; p=0.011), whereas EA patients demonstrated higher cytoplasmic HSET expression (p=0.023). Altogether, these data suggest that nuclear HSET is exclusively associated with African American ethnicity within TN patients.

Nuclear HSET expression is a negative prognostic indicator within African American patients in both univariate and multivariate analyses: Given the marked associations between nuclear HSET expression, ethnicity, and disease progression, an evaluation was undertaken to determine whether HSET has a prognostic value that extends beyond that TN status in AA patients. Thus, the prognostic value of HSET within the AA cohort alone (n=149) was examined. Within the set of AA patients, only nuclear HSET expression (and not cytoplasmic or total) was found to be associated with survival outcomes in univariate analysis. Without adjusting for other factors, higher nuclear HSET expression was associated with worse overall survival (HR=1.41, p=0.008), progression-free survival (HR=1.33, p=0.018), and metastasis-free survival (HR=1.37, p=0.011), as shown in Table 7:

TABLE 7
Correlation of HSET nucleus weighted index with overall, progression-free, and metastasis-free survival
(OS, PFS, and MFS, respectively) within the African American patient sample.
HR = hazard ratio, CI = confidence interval

| Analysis | OS: HR (CI) | P | PFS: HR (CI) | P | MFS: HR (CI) | P |
|---|---|---|---|---|---|---|
| Univariate | 1.41 (1.09, 1.81) | 0.008 | 1.33 (1.05, 1.69) | 0.018 | 1.37 (1.08, 1.75) | 0.011 |
| Multivariate | 1.56 (1.14, 2.13) | 0.006 | 1.44 (1.08, 1.91) | 0.012 | 1.44 (1.07, 1.92) | 0.015 |

These associations were found to be even stronger in multivariate analysis, with higher nuclear HSET predicting worse overall survival (HR=1.56, p=0.006) when adjusting for NPI, stage, TN status, and age; progression-free survival (HR=1.44, p=0.012) when adjusting for stage, TN status, and age; and metastasis-free survival (HR=1.44, p=0.015) when adjusting for stage and TN status (Table 6). Consequently, HSET appears to be a much better prognostic indicator for AA breast cancer patients than the EA population of breast carcinoma patients even after adjusting for age along with TN status and other tumor characteristics. Nuclear HSET was a better prognostic indicator than TN status, which was not a statistically significant prognostic indicator for overall, progression-free, or metastasis-free survival in univariate analysis, and a more significant indicator than tumor size.

### Example 4

### Characterization of Mitotic Arrest (MA) Induced by Centrosome Declustering Drugs

To evaluate the impact of putative declustering drugs on cell cycle progression and hypodiploidy (<2N DNA content, which may indicate apoptotic cells), MDA-MB-231 (231), PC3, and HeLa cells were treated with different concentrations of declustering drugs, stained with propidium iodide, labeled with anti-MPM2 antibody, and then assessed by flow cytometry at multiple time points over 48 h. The chosen cell lines displayed different levels of endogenous centrosome amplification (CA). 231 cells (mutant p53) exhibit high levels of CA (˜20-45%) compared with PC3 (p53 null) and HeLa (wild-type but E6-inactivated p53), which have low basal levels of CA. Consistent with previous reports, the data showed that all drugs induced sustained MA (at least 2× mitotic cells compared with untreated control cultures) at the concentrations indicated. The duration, highest degree, and rapidity of onset of MA varied between drugs, drug concentrations, and cell lines (FIGS. 2A, 2B). In general, the maximum MA achieved was less pronounced in Nos- and PJ-treated cells (FIGS. 2A, 2B). Drug-induced onset of MA was corroborated by substantial increases in cyclin B1 levels in all cell lines (FIG. 2C). For most cases, prolonged MA (˜24 h in duration) was followed by a substantial increase in the subG1 population fraction (FIGS. 2A, 2B). In all cases, significant increases in cleaved caspase-3 over controls was observed (FIG. 2C), suggesting apoptosis. Instances where the subG1 fraction was elevated without cleaved caspase-3 may either represent caspase-independent cell death or the presence of hypodiploid cells whose fate is unclear.

In general, no consistent associations between the extent, duration, or timing of MA within drugs or across cell lines was found (see FIG. 3). In order to discern trends in the metrics of the MA induced by declustering drugs across all cell lines, a more exhaustive evaluation of the impact of peak MA (or “highest reached,” HR), onset of peak (or “time reached highest,” TRH), and duration (sum or total of consecutive time points, CTP, maintaining MA) on subG1 fraction, categories were created for these metrics. Across cell lines, PJ was the fastest-acting in terms of induction of peak MA, as its mean peak onset (MA:TRH) occurred sooner (around “2,” representing 12 h) than those of the other drugs; however, the highest reached MA (MA:HR) was generally smaller than those of the other drugs (FIG. 3). For the other drugs across cell lines, the mean time to peak MA was around “4,” indicating 18 h. RBN generally induced the greatest peak MA (near “4,” representing ≧30% of cells in MA) and also induced the greatest metrics for MA:totCTP (the sum of consecutive time points [CTP] with a certain level of MA, thus serving as a measure of both strength and duration of MA). BN measures of MA:HR and MA:totCTP were similar to those for RBN, although somewhat smaller (FIG. 3). These data suggest that centrosome declustering drugs under study could potentially be functioning via mechanistically distinct pathways to trigger MA and determine cell fate.

### Example 5

### Declustering Drugs Induce CA in Cancer Cell Lines

Given that brominated noscapine (RBN) increases the expression of Plk4, a mediator of CA, other declustering drugs were investigated to determine their effect on expression of PLK4 along with two other mediators of CA, Cyclin E and Aurora A. All of the drugs studied were found to increase expression of PLK4, Cyclin E and Aurora A compared with untreated cultures (FIG. 4). Consequently, CA was assessed in cultures treated with different concentrations of declustering drugs for 6, 12, 18, or 24 h and untreated controls via microscopy. Centrosomes were identified by γ-tubulin and centrin-2 colocalization at discrete foci. Interestingly, all drugs tested induced CA in a statistically significant manner in at least one cell type and drug concentration (10 or 25 μM for all drugs except GF, which was used at 25 and 50 μM). The average percentages of CA over 24 h and the associated fold increases over controls are shown in FIG. 5, respectively. The peak percent CA detected over 24 h is shown in FIG. 6 (only statistically significant (P<0.05) increases over control values are represented in the Figures). Representative confocal micrographs of CA in interphase and mitotic cells, both control and drug treated, are depicted in FIG. 7. No significant correlations between the degree of CA (FIG. 5A) and the expression levels of PLK4, Cyclin E, and Aurora A were found (FIG. 4).

When analyzing correlations between the upregulation of key molecular markers of CA and the extents of drug-induced CA, no significant correlations between the degree of CA (FIG. 5A) and the expression levels of PLK4, Cyclin E and Aurora A (FIG. 4) were found. For example, even though 25 μM Nos caused a surge in the expression levels of Cyclin E and PLK4 in 231 cells (FIG. 4), it failed to induce significant CA in these cells (FIGS. 5A, 5B). Similarly, 25 μM RBN increased Cyclin E and PLK4 expression in PC3 cells but much smaller increases in the expression levels of these proteins in 231 and HeLa cells; nevertheless, RBN induced CA in all three cell lines (FIGS. 5A, 5B).

To better understand the “potency” of drug-induced CA with time, the average fold change in CA over controls over 24 h was assessed (FIG. 5A), whereby the extent of CA across time points (i.e., 6, 12, 18, and 24 h) was averaged and then divided by the extent of CA in control (i.e., 0 h). All of the drugs at least doubled the peak extent of CA in all cell lines tested (FIG. 5A), although the final extent of CA could be small or large in magnitude depending on the initial centrosomal burden as shown in FIG. 4. For instance, although 25 μM PJ treatment resulted in an almost 20-fold increase in peak CA extent in interphase HeLa cells (FIG. 5A), the final extent of CA in this case was rather low at <20% (FIG. 6A). On the other hand, 25 μM RBN only slightly more than doubled the peak CA extent in 231 cells (FIG. 5A) although the final extent of CA was very high (around 90%, see FIG. 6A). These data show that induction of CA is an activity common to all the declustering drugs studied, although the extent of the peak CA induced and its fold difference vary between drugs and cell lines (FIGS. 5A, 6A). Analysis of the CA phenotypes induced by the various declustering drugs showed that RBN stood apart in its ability to potently upregulate centrosome number, which was especially evident in mitotic cells but also present in interphase cells. For example, in 231 cells treatment with 10 and 25 μM RBN resulted in a maximum extent of CA of 56% and 96% in mitotic cells, respectively (FIGS. 5A, 6A), corresponding to ˜2.5- and 2.0-fold increases over controls (FIG. 5A). The extent of CA was less in interphase cells, with maximum values of 31% and 87% for 10 and 25 μM RBN (FIG. 6A). In a similar but more pronounced fashion, 10 and 25 μM RBN also markedly upregulated centrosome numbers in mitotic HeLa cells, with 78% and 42% of cells having CA, representing approximately 40- and 20-fold increases, respectively (FIGS. 5A, 6A). The peak extent in interphase HeLa cells was somewhat less at 30.7% and 48.1% for 10 and 25 μM RBN, respectively (FIG. 6A). For Nos and BN, there was no major difference in CA levels in interphase versus mitotic cells. Since both of these drugs cause mitotic catastrophe in cancer cells, it appears that a comparable level of cell death also occurs in interphase resulting in similar levels of interphase and mitotic cells with CA. For GF and PJ, there was generally more CA in interphase than mitotic cells, which suggests selective elimination of mitotic cells with CA.

Notably, average fold-increases in CA were generally more frequent in interphase cells when compared to mitotic cells (FIG. 5A). The only exception occurred with BN, which demonstrated higher average fold-increases in CA in mitotic cells (FIG. 5A). It is likely that cases where average fold-increases in interphase are substantially greater than in mitosis reflect expeditious elimination of cells with amplified centrosomes via mitotic catastrophe. Similarly, regimens that resulted in a lower average-fold increase in interphase CA compared to mitotic CA may reflect precipitous death of interphase cells with CA. In sum, these data lay the foundation for studying the mechanisms by which declustering drugs induce CA and cell death by providing valuable clues about (i) potencies of CA-inducing activities of these drugs and (ii) the cell cycle phases wherein most cell death induced by these drugs may be occurring. Further, these data show that all the centrosome declustering drugs in the present study are also centrosome amplifying drugs, depending on the cell line and concentration.

As shown in Table 8, compared to HeLa and PC3 cells, 231 cells (which exhibit the greatest endogenous CA among controls, approximately 20-30% on average) were most susceptible to declustering drugs in general:

Table 8. Peak subG1 Percents Over 48 h for Each Cancer and Non-Malignant Cell Line by Drug and Concentration

[symbol]

This is corroborated by the fact that 231 cells exhibited the greatest peak subG1 fraction across cell lines and drugs (25× control after treatment of 231 cells with 25 μM RBN, vs. 9× for HeLa and 8× for PC3, both treated with 10 μM RBN). Within drugs and across cell lines, BN was most effective in 231 cells, (the maximum subG1 fraction was 9.3× control, vs. 4.4× for HeLa and 9.2× for PC3, all treated with 25 μM BN), as was PJ (the maximum subG1 fraction was 10.4× control after treatment with 25 μM PJ, vs. 7.9× control in PC3 cells treated with 25 μM PJ and 4.8× control in HeLa cells treated with 10 μM PJ) (Table 8). GF was most effective in PC3 cells (the maximum subG1 fraction 16.3× control after treatment with 50 μM GF, vs. 6.4× for 231 cells treated with 25 μM GF and 4.3× for HeLa cells treated with 50 μM GF, although these cells do not have substantial endogenous CA (approximately 3% interphase and 4% mitotic CA on average. Altogether, it appears that certain declustering drugs (namely, RBN, BN, and PJ) may be more effective against cancer cell lines with endogenous CA, whereas the efficacy of other agents (namely, GF and Nos) may depend less on endogenous CA.

The above data indicate that RBN, BN and PJ appear to be most effective in 231 cells. To test whether higher susceptibility of 231 cells to these three drugs is related to the extent of drug-induced CA in these cell lines, the average fold-increase in CA (compared to untreated controls) induced by RBN, BN and PJ in 231 cells was evaluated and compared to the average fold-increases in CA induced by these drugs in PC3 and HeLa cells (FIG. 5B). Interestingly, the average fold-increase in CA (compared to untreated controls) in 231 cells is not greater than the average fold-increase in CA induced by these 3 drugs in PC3 and HeLa (in fact, it is significantly lower in 231 compared to PC3 and HeLa) (FIG. 5B). Therefore, it was concluded that the average fold-increase in CA is not responsible for the higher vulnerability of 231 cells to RBN, BN and PJ than PC3 and HeLa cells.

Upon treatment with RBN, BN, and PJ, the final total centrosomal burden (the percent of cells with CA, regardless of cell cycle stage) is much higher in 231 cells as compared to HeLa and PC3 cells (FIG. 5B). This may be attributed to the fact that 231 cells start off with higher centrosome numbers than PC3 or HeLa cells. Since little is known about the biological threshold for total centrosomal load that may overcome the cell's coping mechanisms and tip the cell's fate into apoptosis, one cannot rule out the possibility that the total cellular centrosomal load (resulting from endogenous plus drug-induced CA) may be a key contributor making 231 cells more vulnerable to these drugs than PC3 and HeLa. Taken together, these observations suggest that high levels of endogenous CA in 231 cells may render them more susceptible to RBN, BN, and PJ. By contrast, PC3 and HeLa cells, which lack substantial endogenous CA, are more vulnerable to treatment with GF and Nos.

### Example 6

### Centrosome Amplification in Non-Malignant Cell Lines

To determine whether the CA-inducing activity of declustering drugs is restricted to cancer cells, two non-malignant cell lines, mammary fibrocystic (MCF10A) cells and adult human dermal fibroblasts were treated with these drugs. Neither one of RBN, GF or PJ induced CA or cell death (Table 8) in these cell lines. Specifically, an analysis of the CA phenotypes produced by declustering drug treatment of MCF10A and HDFs showed that neither concentrations of Nos or BN significantly increased CA over control levels in interphase or mitotic MCF10A cells at any time point assessed over 24 h. However, both concentrations of RBN significantly increased the peak extent of CA in interphase and mitotic MCF10A cells (p<0.001 for all, FIG. 6B, which only represents trials that resulted in statistically significant increases in peak CA over controls). 25 μM PJ also increased the peak extent of CA in interphase and mitotic cells (p<0.001 and p=0.002, respectively), while 10 μM PJ induced only a slight increase in the peak extent of CA in interphase cells (8% of cells, p=0.029) (FIG. 6B). 25 and 50 μM GF both increased peak interphase CA (13-16%, p<0.001 for both) although no increase was observed in mitotic cells. Similar to MCF10As, HDFs exhibited only low levels of CA in both interphase and mitotic cells (both approximately 4%). As in MCF10As, Nos and BN did not significantly increase the extent of CA over controls at any of the concentrations or time points assessed. PJ also had no significant impact on CA in HDFs, in contrast to its effect on MFC10A cells. In comparison, 10 and 25 μM RBN increased peak CA over controls in interphase cells (p<0.001 and p=0.001, respectively), with the lower concentration dramatically augmenting peak CA to 56% of cells versus 15% for the higher dose (FIG. 9B). Only 10 but not 25 μM RBN increased the extent of CA in mitotic cells, and this upregulation was only slight (10%, p=0.041). 25 and 50 μM GF also increased peak CA in interphase cells only and to similar extents (14-15%, p<0.001 for both concentrations, FIG. 6B).

Importantly, a therapeutic window exists for several of these agents at the concentrations and in the cell lines tested compared to cancer cells. Nos, BN, and PJ did not cause a significant increase in peak subG1 percent compared to controls (Table 8). RBN and GF did increase peak SubG1 in MCF10A cells compared to controls (p<0.01 for all). However, 10 μM RBN induced a smaller peak subG1 in MCF10A cells as compared to 231 cells (p<0.001), although the same was not true for PC3 and HeLa cells (Table 8). By contrast, increasing the dose of RBN to 25 μM, which caused slightly increased toxicity to MCF10A cells, resulted in much greater increases in toxicity to 231 and PC3 cells (p<0.001). These data suggest that for RBN, even in in vitro cell cultures, a therapeutic window exists and can be exploited to selectively target cancer cell lines. Interestingly, previous work has demonstrated cancer selectivity of RBN in nude mice carrying human ovarian cancer xenografts. In those previous experiments, RBN inhibited tumor progression by inducing apoptosis in tumor cells, but toxicity was not detected in normal tissues. All cancer cell lines were found to be more susceptible to 25 μM GF than MCF10A cells (p<0.001). When the concentration was increased to 50 μM, however, MCF10A and PC3 cells were equally susceptible to the GF, although 231 and HeLa cells remained more susceptible (p<0.001).

In HDFs, all the drugs tested increased peak subG1 over controls in a significant fashion (p<0.01 for all) (Table 8). Nevertheless, for Nos and PJ, both concentrations caused more death in all cancer cell lines vs. HDFs (p<0.001 for all). For BN, the same was true for 231 and PC3 cells (p<0.05 for all) but not HeLa cells, in which there was no significant difference. For GF, both concentrations caused more death in 231 and HeLa cells (p<0.001 for all) but not PC3 cells, in which there was no significant difference. For RBN, both concentrations caused more death in 231 cells and 25 μM RBN caused more death in PC3 cells as compared to HDFs, (p<0.001 for all), but the same was not true for both concentrations in HeLa or 10 μM RBN. Thus, it appears that there may be clinically relevant therapeutic windows for these drugs depending on the type of cancer and the drug dosage.

Altogether, although centrosome declustering drugs induced MA, significant differences existed in the (i) extents and durations of MA, (ii) the size of the subG1 population, (iii) the rapidity of the onset of MA and hypodiploidy, and (iv) the extent to which hypodiploidy was accompanied by caspase-dependent apoptosis (FIGS. 7A-7B) even within a given cell line. In summary, all the centrosome declustering drugs studies were also found to function as centrosome-amplifying drugs, depending on the cell line and drug concentration.

### Example 7

### Effect of Declustering Agents on Centrosome Declustering and Spindle MP

The declustering drugs were further evaluated to determine the extent to which they induce MP. MP was considered low grade if there were only 3 or 4 spindle poles and high grade if there were ≧5 poles. All of the declustering drugs, at one or both concentrations, induced spindle MP in at least one cell type above control levels (FIG. 7A). Several of the drugs induced acentrosomal or ‘acentriolar’ poles (wherein at least one spindle pole stained positively for γ-tubulin but not centrin-2; FIG. 7A), a phenotype not previously reported for these particular drugs. This phenotype has been reported following knockdown of HSET. Also, acentriolar poles were more readily induced in HeLa than in PC3 or 231 cells (FIG. 7A). The mechanism undergirding this phenotype is presently unknown. These observation support the notion that some of the forces that tether together supernumerary centrosomes may also preserve spindle pole integrity.

The declustering agents were further evaluated to determine the extents to which they induced declustering. This analysis shows that the extent of total declustering (the percentage of cells with amplified centrosomes in which no centrosomes were clustered) induced by all these drug regimens was the lowest in 231 cells, which have higher endogenous CA (FIG. 7B). By contrast, in HeLa and PC3 cells, which have comparatively low levels of CA, a majority of the amplified centrosomes were found to be totally declustered (FIG. 7B). For comparison, drug-induced MP, declustering, and acentrosomal pole formation in non-malignant cell lines was assessed (FIGS. 8A-8B). In this case, RBN, GF, and PJ were found to significantly induce MP over control levels, and the supernumerary centrosomes induced tended be declustered.

Thus, it appears that the drugs tested largely induce spindle MP in a declustering-independent manner. Declustering drugs may therefore prove effective in cancers regardless of the extent of CA present.

### Example 8

### Cross Talk Between Drug-Induced Spindle MP, Declustering, and Drug Efficacy

Associations between drug-induced spindle MP, centrosome declustering, and drug efficacy (subG1 extent) were probed in order to identify the phenotypes that contributed most to cell death. Beta regression (a statistical methodology more appropriate for proportions data than linear regression when very low or high percentages are observed) was used to analyze correlates of peak subG1. For this technique, pseudoR² (the squared correlation of linear predictor and link-transformed response) is reported rather than R² as in linear regression, and it indicates the goodness-of-fit of the model.

By this analysis, peak MP was found to significantly correlate with peak subG1 (P=0.00840, pseudoR²=0.321) across all drugs and cell lines, suggesting that generation of spindle MP is a shared mechanism whereby declustering drugs trigger cell death. Importantly, no significant associations between CA and spindle MP were found, consistent with the result that declustering drugs appear to induce spindle MP by disrupting spindle pole and/or centrosome integrity, which in some cases may also decluster centrosomes if an excess is present. Within 231 cells, an even stronger, positive correlation with a very good fit between peak high-grade MP and peak subG1 was found (FIG. 9A; P=0.006; pseudoR²=0.833), underscoring that a desirable attribute for declustering drugs is the ability to induce high-grade rather than low-grade MP. Further, a model including both peak high-grade and low-grade MP together was found to be better in predicting peak subG1 (P=0.001; pseudoR²=0.860) (FIG. 9B). Specifically, within this model, the prediction of peak subG1 using peak high-grade MP was very highly statistically significant (P<0.00001) and the beta coefficient was positive, indicating a positive correlation between peak high-grade MP and subG1 generation. The prediction of peak subG1 using peak low-grade MP was very highly statistically significant (P=0.00001), and the beta coefficient was negative, indicating a negative correlation between peak low-grade MP and peak subG1. This is consistent with the notion that high-grade MP engenders intolerably severe aneuploidy that is likely to culminate in cell death, whereas low-grade MP is more likely to be survivable and perhaps advantageous to cancer cells. Clear trends were not uncovered for centrosome declustering and subG1 across drugs, although one cannot rule out its importance within individual drugs, as the number of data points for peak subG1 was limiting.

In HeLa cells, peak MP (any grade) positively correlated with peak subG1 (P=0.0055; pseudoR²=0.575; FIG. 9D). Also, peak high-grade MP positively correlated somewhat with peak subG1 (P=0.028; pseudoR²=0.271; FIG. 9E). Notably, the peak acentriolar pole percentage positively correlated with peak subG1 (P=0.0023; pseudoR²=0.600; FIG. 9F), so daughter HeLa cells without centrosomes may be inviable. Indeed, based on the pseudoR² value, peak acentriolar pole formation was superior to all other variables in predicting peak subG1. Peak total declustering also positively correlated with peak subG1 (P=0.020; pseudoR²=0.424; FIG. 9G), strengthening the idea that more extensive declustering kills more cancer cells.

In PC3 cells, no association between peak MP and peak subG1 across drugs was found. However, when analyzing the correlation between the average fold increase in CA induction with peak subG1 percent, an interesting trend emerged. Specifically, in PC3 cells, the proportion variable (peak subG1) always lay within the 30-70% range and the other variable (fold increase in CA) was continuous; therefore a linear regression was implemented for analysis. This analysis showed that the average fold increase in CA in interphase positively correlated with peak subG1 (P=0.057; R²=0.619; FIG. 9C), suggesting that an increase in CA may promote cell death.

MCF10A cells and human dermal fibroblasts were further evaluated to study the impact of treatment with declustering drugs on spindle MP and subG1 induction in non-transformed cells. In both of these cell types, peak MP positively correlated with peak subG1 (R²=0.82 with P=0.003 and R²=0.89 with P<0.001, respectively), suggesting that MP is also toxic to normal cells.

### Example 9

### HSET is Overexpressed in a Variety of Human Cancers

Given the crucial requirement of centrosome clustering mechanisms for the viability of cancer cells with extra centrosomes, the abundance of the clustering protein HSET in various cancers harboring extra centrosomes was investigated. Upregulating HSET expression may provide a means to permit clustering of extra centrosomes and may facilitate maintenance of low-grade aneuploidy so as to foster cell viability and allow malignant transformation and tumor evolution to proceed. An in silico gene expression analysis using publically available microarray data was employed to determine the expression level of HSET in various cancer tissue types. One-channel microarray data for glioblastoma, leukemia, lung and breast cancer patients with their normal sample pairs were collected from Gene Expression Omnibus (GEO) database. Each of these samples was then Robust Multiarray (RMA) normalized, and their logarithm to base 2-transformed HSET gene expression values were plotted to determine the difference as shown in FIG. 10A-F. Differences in HSET gene expression for cancer and normal sample groups were determined using a two-tailed hypothesis test. The statistical results indicated higher HSET gene expression in glioblastoma, leukemia, lung, breast, colon and cervical tumor samples as compared to their corresponding normal tissues. The average HSET expression for glioblastoma (n=20) and colon cancer (n=53) patients was found to be ˜3-fold higher than normal samples (n=3 and 10, respectively) (p<0.005), followed by breast cancer patients (n=179) with more than 5-fold higher expression in tumors than in normal samples (n=16) (p<0.001). The in silico results were consistent with observations from a previous study wherein HSET mRNA expression was significantly elevated in a broad panel of primary tumor tissue compared to corresponding normal tissue. The in silico data corroborates immunohistochemical analysis suggesting significantly higher HSET expression in glioblastoma, colon and cervical tumors (FIGS. 10J, 10K, 10L) as compared with their respective adjacent normal tissue samples (FIGS. 10G, 10H, 10I). These data suggest HSET OE is a general feature of cancers exhibiting significant centrosome amplification.

### Example 10

### HSET is Overexpressed in Human Breast Cancers

The in silico analyses of microarray data showed that breast cancers display significantly higher HSET expression (˜5-fold) than corresponding normal tissue. Further, given the pronounced occurrence of amplified centrosomes and centrosome clustering in aggressive breast cancer, HSET was further evaluated to determine whether a role in tumor progression was wholly dependent on its known function of clustering supernumerary centrosomes. Accordingly, 16 fresh-frozen human tumor samples were immunoblotted along with their paired adjacent normal tissues for HSET. An enhanced expression of HSET was observed in 10 tumor samples compared to their normal adjacent tissues; seven representative normal/tumor sample pairs are shown in FIG. 11A. The remaining 6 normal/tumor pairs showed negligible HSET OE (data not shown). Additionally, HSET expression in most human breast cancer cell lines was much higher than in non-cancerous or pre-malignant cell lines such as NIH3T3 and those of the MCF10 series (MCF10A, MCF10AT1, MCF10DCIS) (FIG. 11B), indicating that HSET OE typifies breast cancer cells.

Since higher HSET protein levels could arise either from an upregulation of transcription from the endogenous locus and/or an amplification of the locus encoding HSET, the copy numbers of the locus encoding HSET gene in normal and breast tumor tissues were determined using fluorescence in situ hybridization (FISH) to directly evaluate the HSET copy number per cell in paraffin-embedded breast tumor tissues. Two bacterial artificial chromosome (BAC) probes were hybridized to primary breast tumor tissues, one from the HSET locus on chromosome 6 (RPCI-11 602P21, green) and one from the chromosome 6 centromere (CH514-7B4, red). Amplification of HSET was visualized as an increase in the number of HSET signals relative to the number of control centromere signals. HSET amplification was scored by FISH in four breast tumor tissues; among these, three tumors exhibited HSET amplifications. No amplification of the HSET locus was observed in the normal adjacent tissues in these samples. Various types of copy number changes associated with HSET were observed as shown in FIGS. 11C and 11D. FISH with the centromere probe indicated that most increases in HSET loci were not due to polyploidy of chromosome 6. Rather, only 5% of cells were aneuploid. 500 cells each were counted from 2 tissue samples, and 38% of cells showed 3 or more copies of HSET paired with only 1 or 2 copies of the centromere (FIG. 11D). Cancer cells isolated from fresh human breast tumors also showed HSET amplification (data not shown). These findings indicate alterations in the HSET gene copy number occur during tumorigenesis. HSET gene amplifications in specific breast tumor samples were correlated with increased expression of HSET protein in all those samples using immunoblotting methods (data not shown).

### Example 11

### HSET Overexpression Correlates with Breast Cancer Progression and Aggressiveness

An immunohistochemical staining approach was employed to determine whether HSET overexpression correlates with breast cancer progression and aggressiveness. A total of 60 clinical specimens representing 10 cases each of normal breast, ductal hyperplasia (DH), atypical ductal hyperplasia (ADH), ductal carcinoma in situ (DCIS), invasive breast carcinoma (low-grade) and invasive breast carcinoma (high-grade) were stained. Consistent with the immunoblotting data, this immunohistochemical analysis showed that HSET is selectively overexpressed in human breast cancers with negligible or absent expression in normal breast epithelia (FIGS. 12A-C). In particular, a selective increase in HSET nuclear staining was observed in the tumor samples. Among subtypes based on varying types and extents of intraductal proliferation, a progressive increase in HSET nuclear staining intensity and frequency from ductal hyperplasia (DH) (FIG. 12B) to atypical ductal hyperplasia (ADH) (FIG. 12C) to ductal carcinoma in situ (DCIS) (FIG. 12D) was observed. In invasive breast cancers (both low- and high-grade), HSET nuclear staining was remarkably intense, with a significant increase in the number of positively stained nuclei per field in high-grade cancers (FIGS. 12E and 12F) compared to low-grade ones (FIGS. 12B, 12C, 12D). A majority of normal breast tissue samples (85%) showed no staining for HSET, while the remainder showed very weak staining (FIG. 12A, data not shown). A weighted index (WI) for HSET expression as the product of the staining intensity score (0, 1, 2, or 3) and the percentage of positive nuclei for each sample was calculated. The HSET WI serves as an independent measure of the strength of HSET protein expression across all breast tumor specimens. Nuclear HSET WI values were then correlated within normal and tumor samples and also within the grade of tumor samples. Interestingly, nuclear HSET WI showed a strong correlation with increasing tumor grade in breast cancer (FIGS. 12G, 12H). Collectively, these observations indicate robust HSET overexpression in human breast tumors suggesting that abnormal HSET levels correlate with breast cancer development and that HSET might play a role in the progression of tumors into more malignant and aggressive forms.

Having established a significant correlation between HSET expression and tumor differentiation, it was of interest to investigate a possible association of nuclear HSET WI with progression-free survival (PFS) and overall survival (OS) in breast cancer patients, whereby PFS was calculated as the number of days from diagnosis to the first local recurrence or metastasis if one occurred or the last follow-up if the patient did not progress, and OS was calculated based on the number of days from diagnosis to death or last follow-up if death was not recorded. Nuclear HSET WI was also categorized into high and low groups based on the median. Irrespective of the receptor status (for n=163 patients), those with higher nuclear HSET WI (shown as HSET WI positive in FIGS. 12I, 12J) had statistically shorter PFS (p=0.0034) and OS (p=0.0412) than patients with lower nuclear HSET WI (shown as HSET WI negative in FIGS. 12I, 12J), clearly demonstrating that higher nuclear HSET expression levels significantly correlate with poorer clinical outcomes.

### Example 12

### HSET Overexpression is Associated with Enhanced Cell Proliferation

Since elevated HSET expression exhibits a strong correlation with the development and progression of cancer, it was of interest to determine whether high HSET levels had any impact on the kinetics of cancer cell proliferation in vitro. To this end, HeLa cells stably transfected with HSET-GFP were evaluated to examine and compare the levels of various cell proliferation markers in HeLa-HSET-GFP and HeLa cells. Immunoblot analysis revealed that Ki67 levels were substantially elevated in HeLa-HSET-GFP cells compared with wild-type HeLa cells (FIG. 13A). This finding was consistent with the strikingly high Ki67 labeling index observed in HeLa-HSET-GFP cells via immunostaining (FIG. 13B), and is noteworthy since the Ki67 labeling index often correlates with the clinical course of cancer. Essentially, the proportion of Ki67-positive cells in a cell population has strong prognostic value and may predict tumor recurrence in cancer patients. Immunofluorescence staining for BrdU, a marker for cells undergoing S phase, also showed that a greater proportion of HeLa-HSET-GFP cells were BrdU-positive compared with wild-type HeLa cells (FIG. 13C). A visual quantitation of these observations revealed significantly elevated Ki67 and BrdU incorporation in HeLa-HSET-GFP cells as compared with HeLa cells (FIG. 13D). Enhanced Cdk1 activity and higher expression of phosphorylated histone-H3 in HeLa-HSET-GFP cells was seen compared with HeLa cells, which is indicative of a larger proportion of cells in the HeLa-HSET-GFP cell line undergoing mitosis (FIG. 13A). Taken together, this evidence strongly supports a pro-proliferative role for HSET overexpression in the cellular context of cancer cells. HeLa-HSET-GFP cells also displayed significantly enhanced cell proliferation capacities when compared with wild-type HeLa cells in a Trypan Blue assay. Equal numbers of each cell type were seeded on day 0 and were allowed to grow for 2 days (48 h), and the number of cells were counted using Trypan Blue. Based on the data, the doubling time of HeLa-HSET-GFP cells was found to be ˜11 h as compared with ˜16 h for HeLa cells (FIG. 13E).

Colony formation assays with HeLa cells transiently transfected with control vector (CV), HSET-GFP plasmid (OE) or HSET-GFP siRNA (KD) were also performed. HSET overexpressing (OE) cells were able to form a significantly greater number of colonies as compared with cells transfected with control vector (CV). Much fewer colonies were observed following transfection with the HSET knockdown plasmid, HSET-GFP siRNA (KD). Similar proliferation effects were confirmed by colony formation assay in another breast cancer cell line, MDA-MB-231, following transient transfection with HSET OE and KD (FIG. 14). Taken together, these data demonstrate that HSET overexpressing cells exhibit enhanced cell proliferation, which may confer significant proliferative advantages to cancer cells.

### Example 13

### HSET Overexpression Leads to Accelerated Cell Cycle Kinetics

Since HSET overexpression enhances cellular proliferation in HeLa cells, changes in the cell cycle kinetics was investigated in cells stably overexpressing HSET (HeLa-HSET-GFP cells) as compared with the parental ones. To this end, HeLa and HeLa-HSET-GFP cells were synchronized using a single thymidine block (19 h) followed by flow cytometric analysis of cell cycle profiles of HeLa-HSET-GFP and HeLa cells upon their release from a G1/S block. DNA content was analyzed with propidium iodide (PI) staining, in which the G2/M population was represented by double the intensity of PI (4N) compared with the G1 cell population (2N). Anti-MPM-2 antibody tagged with Alexa-488 secondary antibody was used to detect a mitosis-specific marker (MPM-2), in order to distinguish between 4N DNA-bearing G2 and M populations. Close interval cell cycle profiling revealed that HeLa-HSET-GFP cells demonstrated faster cell cycle progression kinetics; in other words, the duration of one complete cell cycle was reduced in HSET-transfected cells (10.5 h) as compared with wild-type cells (13 h), with a stark shortening of the G2 and M phases (FIGS. 15A, 15B, 15C). This trend was reflected when cyclin B1 levels (indicating mitotic phase) were followed in synchronized HeLa and HeLa-HSET-GFP cells using Western blotting. While cyclin B1 levels surged at 10 h followed by a decline in HeLa cells, they peaked at 8 h and then declined in HeLa-HSET-GFP cells (FIG. 15D). Transient knockdown (KD) of HSET in HeLa cells resulted in a marginal decrease in cell cycle duration (14 h as compared to 13 h in HeLa cells) with a protracted G2/M phase (FIGS. 16A-C). This observation is in accordance with the previous finding that HSET depletion in human fibroblasts leads to delayed cyclin A degradation.

In view of the significant contribution of the G1 phase to cell cycle duration, the effect of HSET overexpression (OE) and HSET knockdown (KD) on G1 phase kinetics was investigated. Upon gradual lowering of the serum concentration from 10% to 0% over 24 h and an additional 12 h serum starvation, HeLa cells transiently transfected with control vector (CV), HSET overexpressing plasmid (OE) and HSET knockdown vector (KD) were replenished with serum-containing medium and stained with a cell-clock dye (a redox dye that changes color corresponding to distinct cell cycle phases) in a Cell Clock™ Assay (Biocolor, UK). Yellow cells in the culture represent G1 phase cells, and their color changes to light green in S phase. This allowed for monitoring of the proportion of G1 (yellow-colored) cells from 0 h (50-70% G1enrichment) to 9 h after serum replenishment in all three cases (CV, OE and KD). Negligible differences in the proportion of G1 cells among all three conditions was observed (FIGS. 16A-C). This suggests that unlike G2 and M phase kinetics, the kinetics of G1 phase are not significantly affected by HSET overexpression (OE).

Faster kinetic progression of HeLa-HSET-GFP cells (through G2 and M) compared with HeLa cells raises the possibility that G2/M or spindle assembly checkpoint (SAC) functions may be compromised in HeLa-HSET-GFP cells. Mad1 is a critical component of the SAC along with Mad2, and an imbalance in the Mad1/Mad2 protein ratio results in a damaged SAC permitting premature anaphase entry and chromosome instability. Interestingly, HeLa-HSET-GFP cells were found to express markedly higher levels of Mad1 with a distinct nuclear envelope localization compared with parental HeLa cells (FIGS. 17A, 17B). Given the known association of HSET with importins, this observation indicates that HSET might be involved in regulating mitotic entry and export. By contrast, there was no significant difference in the levels of Mad2 between the two cell lines (FIG. 17A), showing that the Mad1/Mad2 balance is perturbed in HSET overexpressing HeLa cells. These data support the notion that excess HSET directly or indirectly incapacitates the SAC by disrupting the Mad1/Mad2 balance, such that HeLa-HSET-GFP cells proceed rapidly through the cell cycle in the presence of compromised checkpoints, increasing the likelihood of generating aneuploidy and accelerating the process of tumor progression and evolution.

The data from the HeLa-HSET-GFP cells demonstrates that HSET overexpression (OE) can accelerate the kinetics of G2 and M phases (FIGS. 15A, 15B, 15C). Intriguingly, the immunohistochemical data from clinical tumor samples (FIG. 18) showed strong nuclear localization of HSET. To further examine how elevated HSET levels may hasten progression through G2 and M phases and to exclude the possibility that faster cell cycle kinetics may result from artifactual mislocalization of HSET, the sub-cellular localization of HSET in HeLa cells at various cell cycle stages was examined. The observation that HSET is conspicuously confined to the nucleus throughout interphase (FIG. 19) is consistent with the finding that XCTK2, the Xenopus homolog of HSET, is sequestered in the nucleus during interphase in a Ran-dependent manner via the association of the NLS of XCTK2 with importin α/β. In summary, HSET nuclear localization in interphase strongly suggests that the acceleration of the kinetics of G2 may be ascribed to a hitherto unknown activity of HSET within the nucleus.

### Example 14

### HSET Overexpression Upregulates Survival Signaling in Cancer Cells

While the rate of cellular proliferation dictates the number of tumor cells and tumor growth, cell survival and/or apoptosis pathways also have a significant bearing on overall tumor growth. Having found that HSET overexpression (OE) can enhance the kinetics of cell proliferation in tumors, it was of interest to determine whether elevated HSET levels have any impact on the status of pro-survival signaling in HeLa cells. Immunoblots showed enhanced survival signaling as evidenced by notably high survivin and p-Bcl2 levels in HeLa-HSET-GFP cells (FIG. 17C) compared with the levels seen in parental HeLa cells. To investigate whether HSET overexpression affects signaling pathways that impinge on cell proliferation, adaptation to hypoxic environments, or cell survival in breast cancer cells, levels of key proliferation, hypoxia and cell survival markers were investigated in parental MDA-MB-231 cells, in MDA-MB-231-HSET overexpressing cells and in MDA-MB-231-HSET knockdown (KD) cells. Significantly enhanced levels of survivin and phospho-survivin, hypoxia-induced factor HIF1α, SAC protein Mad1 and the mitotic kinase Aurora B were observed in MDA-MB-231-HSET overexpressing cells (FIG. 17D). However, upon expression of HSET siRNA from the HSET knockdown (KD) vector, marginal or no reduction was observed in the expression levels of these proteins as compared to their respective levels in control cells (FIG. 17D). The differential effects observed upon HSET overexpression (OE) and knockdown (KD) indicate that HSET may not normally be a key regulator of proliferation and survival pathways. Several studies have in fact shown that HSET function is dispensable for the viability of non-cancerous cells. However, the overexpression data strongly suggest that elevated HSET levels thrust proliferation and survival signaling in cancer cells into an “overdrive” mode. In sum, while HSET plays a non-essential role in regulating survival signaling in cancer cells, HSET overexpression enhances both the proliferation as well as the survival of cancer cells and perhaps fuels tumor progression by providing cancer cells with a proliferation and survival advantage. These data indicate that cancer cells may employ auxiliary pathways/mechanisms, such as those involving the kinesin motor HSET, to their advantage.

To further explore the physiological role of HSET in cell survival signaling, the ability of MDA-MB-231 cells to resist UV-induced apoptosis was examined. Briefly, MDA-MB-231 cells were transiently transfected with a control vector (CV), HSET overexpression (OE) construct or an HSET knockdown (KD) construct expressing HSET siRNA (˜70% transfection efficiency) 24 h prior to UV irradiation. Following a 10 min exposure to UV-C at 25 J/m², cells were placed in the incubator for apoptosis induction for 5 h. Lysates were then collected for determining HSET and cleaved caspase-3 (an early marker for apoptosis induction) protein levels, and cell viability was determined using a Trypan Blue assay. Western blot analysis revealed significantly higher cleaved caspase-3 induction in cells with HSET KD, whereas cells with HSET OE showed slightly lower cleaved caspase-3 levels as compared with cells transfected with control vector (CV) (FIG. 17E). These data reflect the ability of HSET OE to promote cell survival in cancer cells.

### Example 15

### HSET Overexpression Increases Steady-State Survivin Levels by Decreasing its Poly-Ubiquitination

Given the extensive upregulation of survivin protein expression upon HSET OE and significant reduction upon HSET depletion, it was of interest to determine if HSET occurs in the same protein complex as survivin and whether HSET overexpression has any effect on the APC/C-dependent proteolysis of survivin. Accordingly, co-immunoprecipitation analysis was undertaken to determine if HSET and survivin co-immunoprecipitate with each other. HSET was immunoprecipitated from whole cell lysates of MDA-MB-231 cells carrying (i) a control vector (CV), (ii) an HSET OE plasmid or (iii) an HSET siRNA-bearing construct (KD). Immunoblots of these immunoprecipiates probed for survivin confirmed that the anti-HSET antibody was able to pull down survivin in all the three cases, with an increased survivin pull down in cell lysates overexpressing HSET (FIG. 17F). This association was further confirmed by immunoprecipitating survivin and then probing with HSET antibody (FIG. 17G). Overall, these data indicate direct or indirect binding of HSET to survivin.

Since the role of survivin in prosurvival signaling is regulated by its degradation via ubiquitination, it was of interest to test the hypothesis that increased HSET binding to survivin protects survivin from ubiquitination and its APC/C-dependent degradation. In MDA-MB-231 cells transiently transfected with control vector (CV), HSET-GFP plasmid (OE) or HSET siRNA plasmid (KD), anti-survivin antibody was used to pull down survivin immunoprecipitates, which were then immunoblotted for survivin and ubiquitin. Intriguingly, reduced polyubiquitin signals in HSET overexpressing cells were observed, even though survivin was extensively overexpressed in those cells (FIG. 17F) as observed earlier (FIG. 17D). On the other hand, marginally higher ubiquitin levels were observed in HSET knockdown (KD) cells as compared with control cells, even though survivin levels were comparable in both the cases. These observations, in sum, uncover a previously unrecognized role of HSET in supplementing prosurvival pathways to fuel tumor progression.

### Example 16

### Differences in Npap60L Expression Relative to CAS Expression in TNBC Patients of Different Ethnic Background

A key driver of metastasis in people of African descent with TNBC may be a low Npap60L-to-Npap60S ratio owing to which more HSET accumulates in nuclei of African American (AA) TNBCs wherein it activates expression of metastasis-related genes. Table 9 shows differences in Npap60L expression relative to CAS expression in AA TNBC patients compared to European American (EA) TNBC patients, suggesting that the Npap60L-importin-α-KifC1 pathway may be targeted to inhibit metastasis in AA TNBCs. In fact, HSET and Npap60L were co-immunoprecipitated together from breast cancer tissue, indicating that they are both present in the same protein complex in breast cancer cells (data not shown).

TABLE 9
(Yale Triple Negative Breast Cancer Cohort
(GEO ID = GSE46581), AA, n = 43 EA, n = 25; two-tailed t test)

| Statistic | Npap60L | Npap60S | Importin α | CAS | NPAP60L/CAS | Npap60S/CAS |
|---|---|---|---|---|---|---|
| AA mean | 7.317 | 8.991 | 10.659 | 11.981 | 0.609 | 0.752 |
| EA mean | 8.435 | 8.830 | 10.114 | 11.736 | 0.722 | 0.753 |
| p value | 0.009 | 0.706 | 0.274 | 0.252 | 0.003 | 0.977 |

### Example 17

### Prc1 Promotes Nuclear Accumulation of HEST in AA TNBCs

Protein regulator of cytokinesis 1 (Prc1), is a non-motor microtubule-associated protein that has been shown by several groups to be a first degree neighbor of HSET in interactomes. Over 90% of TNBCs overexpress Prc1 (˜10.5-fold greater than adjacent normal breast tissue). In addition, Prc1 is included in the MammaPrint 70-gene signature, which predicts distant metastasis in breast cancer. Higher Prc1 is independently associated with worse distant metastasis-free survival across breast cancer patients, a trend that was upheld in TNBC patients. Prc1 was also elevated in MDA-MB-231 TNBC cells that migrated faster in a transwell assay as compared with those that did not. Table 10 shows Prc1 expression in AA TNBC patients and EA TNBC patients. The significantly higher Prc1 expression in AA TNBC patients suggests that Prc1 might be collaborating with KifC1 to drive more aggressive tumor phenotypes in AA TNBCs. It was also found that HSET and Prc1 colocalize extensively in the nucleus in MDA-MB-231 cells, that HSET and Prc1 mostly localize to the nucleus during interphase and that HSET co-immunoprecipitates with Prc1 (data not shown).

TABLE 10

| | FOXM1 | KIFC1 | PRC1 | PLK1 | CDK1 | AURKA | CDCA8 | KPNA2 | CDK2 |
|---|---|---|---|---|---|---|---|---|---|
| AA mean | 10.1801 | 9.5957 | 10.4446 | 9.3401 | 10.2377 | 9.2269 | 9.1107 | 11.8583 | 9.7 93 |
| EA mean | 9.60254 | 8.94631 | 9.95508 | 8.7588 | 9.55769 | 8.6376 | 8.5267 | 11.4623 | 9.6377 |
| P value | 0.00185 | 4.2E−05 | 0.0005 | 0.0009 | 9.5E−06 | 0.0003 | 0.0001 | 0.00172 | 0.0739 |

(AA, n = 61; EA, n = 624; TCGA dataset, 2-tailed t tests)

The above description is for the purpose of teaching the person of ordinary skill in the art how to practice the present application, and is not intended to detail all those obvious modifications and variations of it that will become apparent to the skilled worker upon reading the description. It is intended, however, that all such obvious modifications and variations be included within the scope of the present application, which is defined by the following claims. The claims are intended to cover the components and steps in any sequence that is effective to meet the objectives there intended, unless the context specifically indicates the contrary. All of the references and patent disclosures cited in the specification are expressly incorporated by reference in their entirety herein.

What is claimed is:

1. A method of assessing the prognosis of a patient diagnosed with triple negative breast cancer, the method comprising:
performing an assay on a biological sample comprising breast cancer cells from said patient to determine whether said breast cancer cells express an elevated level of nuclear HSET; and
providing an assessment of the prognosis of said patient based on the result of said assay, wherein an elevated level of nuclear HSET in said breast cancer cells indicates a poorer prognosis for said patient compared to a patient with triple negative breast cancer expressing a lower level of nuclear HSET.

2. The method of claim 1, further comprising the step of determining expression levels of Npap60L and cellular apoptosis susceptibility protein (CAS) from said patient's biological samples and determining an Npap60L to CAS expression level ratio, wherein a ratio of <0.7 indicates a poorer prognosis for said patient compared to a patient with triple negative breast cancer with an Npap60L to CAS expression level ratio of >0.7.

3. The method of claim 1, further comprising performing an assay on a biological sample comprising breast cancer cells from said patient to determine whether the breast cancer cells express an elevated level of nuclear Prc1, wherein an elevated level of nuclear HSET and Prc1 indicates a poorer prognosis for said patient compared to a patient with triple negative breast cancer expressing lower levels of both nuclear HSET and Prc1.

4. The method of claim 1, wherein said patient is a person of African descent.

5. The method of claim 1, wherein said assay comprises an immunohistochemical analysis of said breast cancer cells.

6. The method of claim 5, wherein said immunohistochemical analysis comprises exposing said cells to an anti-HSET antibody under conditions sufficient to allow said antibody to specifically bind to HSET.

7. The method of claim 1, wherein said assay comprises the steps of preparing a nuclear extract from said biological sample and determining a level of HSET in said nuclear extract.

8. The method of claim 7, wherein said determining step comprises exposing said nuclear extract to an anti-HSET antibody under conditions sufficient to allow said antibody to specifically bind to HSET.

9. The method of claim 1, further comprising the step of determining whether said breast cancer cells express an elevated level of a secondary marker selected from the group consisting of Npap60L, CAS, Prc1, Ki67, survivin, phospho-survivin, HIF1α, aurora kinase B, Mad1, p-Bcl2, FoxM1, Plk1, Aurora A, KPNA2 and combinations thereof.

10. The method of claim 1, further comprising the step of determining whether said breast cancer cells exhibit enhanced Cdk1 activity, or express phosphorylated histone-H3, or both.

11. The method of claim 1, further comprising the step of determining the patient's geographic origin(s) by ancestry analysis of the patient's genomic DNA.

12. The method of claim 1, further comprising the step of administering to said patient an effective amount of a therapeutic agent.

13. The method of claim 12, wherein said breast cancer cells express an elevated level of nuclear HSET and wherein said therapeutic agent comprises an inhibitor of HSET.

14. The method of claim 13, wherein said inhibitor of HSET is a small molecule drug that targets a motor domain of HSET and/or specifically binds to a HSET/microtubule binary complex and inhibits HSET microtubule-stimulated or microtubule-independent ATPase activity.

15. The method of claim 13, wherein said inhibitor of HSET is a declustering agent selected from the group consisting of AZ82, PJ-34, griseofulvin, noscapine, 9-bromonoscapine, reduced bromonoscapine, N-(3-brormobenzyl) noscapine, aminonoscapine, CW069, N2-(3-pyridylmethyl)-5-nitro-2-furamide, N2-(2-thienylmethyl)-5-nitro-2-furamide, N2-benzyl-5-nitro-2-furamide, derivatives and analogs therefrom.

16. The method of claim 13, wherein said inhibitor of HSET is administered in combination with a PARP inhibitor, an inhibitor of the Ras/MAPK pathway, an inhibitor of the PI3K/AKT/mTOR pathway, or a combination thereof.

17. The method of claim 13, wherein said therapeutic agent further comprises an agent that negatively regulates the expression and/or activity of a protein selected from the group consisting of Npap60L, CAS, Prc1, Ki67, survivin, phospho-survivin, HIF1α, aurora kinase B, Mad1, p-Bcl2, FoxM1, Plk1, Aurora A and KPNA2.

18. A kit comprising an HSET binding agent and one or more agents that specifically bind to one or more gene products selected from the group consisting of gene products of Npap60L, CAS, Prc1, Ki67, survivin, phospho-survivin, HIF1α, aurora kinase B, Mad1, p-Bcl2 FoxM1, Plk1, Aurora A, KPNA2 and combinations thereof.

19. The kit of claim 18, wherein said HSET binding agent and said one or more agents are antibodies.

20. The kit of claim 18, further comprising:
(a) one or more reagents for immunohistochemical staining of nuclei; or
(b) one or more reagents for preparation of a nuclear fraction or extract; or
both (a) and (b).
